(19) United States
(12) Patent Application Publication
(10) Pub. No.: US 2011/0137127 A1
(43) Pub. Date: Jun. 9, 2011

(54) Dual screen intubation system

(75) Inventors: John Schwartz, Williamston, MI (US); Richard Schwartz, Evans, GA (US); Harsha Setty, Martinez, GA (US)

(73) Assignee: AI Medical Devices, Inc., Williamston, MI (US)

(21) Appl. No.: 12/928,126

(22) Filed: Dec. 3, 2010

Related U.S. Application Data
(60) Provisional application No. 61/283,691, filed on Dec. 8, 2009.

(51) Int. Cl. A61B 1/267

(52) U.S. Cl. 600/188

(57) ABSTRACT

A system and method are disclosed for using a laryngoscope blade with a camera and an endotracheal intubation device with a camera to enable simultaneous viewing of images from the two cameras on a display means such as an LCD display screen. The system enables accurate insertion and placement of an endotracheal tube in a patient with less risk to the patient.

### CROSS-REFERENCE TO RELATED APPLICATION

Priority is claimed to U.S. Provisional Application No. 61/283,691, filed Dec. 8, 2009, the disclosure of which is incorporated herein in its entirety.

### BACKGROUND

In U.S. Publication No. 2010/0095969 to Schwartz et al. (U.S. Ser. No. 12/587,905, filed Oct. 15, 2009 and published Apr. 22, 2010), an endoscopic intubation device is described which is a suitable intubation device for the system of the present disclosure. The intubation device has a camera at the distal end at a tip of a stylet for viewing the airway passage of a patient during intubation with an endotracheal tube mounted on the stylet.

U.S. Pat. No. 7,458,375, U.S. Pat. No. 7,658,708, U.S. Publication No. 2008/0200761, and U.S. Publication No. 2008/0308098 to Schwartz et al. are directed to endotracheal intubation devices having a curveable portion and internal optics or a viewing device which facilitate the insertion of an endotracheal tube into a patient.

U.S. Publication No. 2008/0208000 to Schwartz et al. is directed to a device for endotracheal intubation and fluid delivery into the trachea of a patient. The fluid delivery device includes a tubular housing adapted to be sealably mounted on an elongate element of the endoscope and delivers a fluid thereto.

U.S. Publication No. 2009/0090357 to Schwartz et al. is directed to a guide/laryngoscope blade device for facilitating the insertion of a medical device into the trachea of a patient.

The foregoing patents and publications are incorporated by reference herein in their entireties.

### OBJECTS

It is an object of the present disclosure to provide a system which enables simultaneous viewing of a patient's airway passage by a camera in the tip of an endoscope/intubation device and in the tip of a laryngoscope blade. It is further an object to provide a system which is also very effective in providing original and safe intubation.

These and other objects will become increasingly apparent by reference to the following description and the drawings.

### SUMMARY

The disclosure generally relates to an endotracheal intubation system adapted for two-handed operation by a medical operator, the system comprising: (a) an endotracheal intubation device comprising: (i) a stylet adapted for mounting an endotracheal tube, the stylet having a proximal end and a distal end, (ii) optionally an operator handle mounted to the stylet at the proximal end of the stylet, and (iii) a first camera means mounted to the stylet adjacent the distal end of the stylet and enabling viewing of a first image of a patient's airway passage on a first view screen; (b) a laryngoscope blade for lifting a patient's epiglottis with simultaneous viewing of the patient's airway passage by the endotracheal intubation device, wherein (i) the laryngoscope blade has a proximal end and a distal end, and (ii) the laryngoscope blade comprises a second camera means mounted adjacent the distal end of the laryngoscope blade, the second camera means enabling viewing of a second image of the patient's airway passage on a second view screen; and (c) a display means for simultaneously receiving and viewing visual images from at least two different camera means, wherein (i) the display means is electronically connected to the first camera means and to the second camera means, and (ii) the display means comprises the first view screen for viewing the first image and the second view screen for viewing the second image, the first view screen being positioned adjacent the second view screen on the display means; wherein during use by a medical operator, a proximal end of the intubation device and the proximal end of the laryngoscope blade are each adapted to be manipulated together in two-handed operation by the operator's left and right hands together.

Various refinements and extensions of the disclosed system are possible. For example, the display means can comprise one or more monitor screens (e.g., LCD screens) adapted to independently and simultaneously display the first image and the second image. The first camera means and the second camera means can be independently selected from the group consisting of a CMOS device and a CCD device. In an embodiment, the display means comprises (i) a first monitor screen comprising the first view screen and being adapted to display the first image, and (ii) a second monitor screen comprising the second view screen and being adapted to display the second image. In another embodiment, the display means comprises a first monitor screen, the first monitor screen comprising the first view screen and the second view screen as discrete regions of the first monitor screen and being adapted to independently and simultaneously display the first image and the second image on the first monitor screen. The electronic connection between the display means and the first camera means and/or the second camera means can be independently selected from a wired electronic connection, a wireless electronic connection, and combinations thereof. In an embodiment, the display means is mounted on the handle of the endotracheal intubation device so that the first view screen and the second view screen are viewable together by the operator during use. In another embodiment, the display means is mounted adjacent the proximal end of the laryngoscope blade so that the first view screen and the second view screen are viewable together by the operator during use. In another embodiment, the display means is mounted on a structure other than the endotracheal intubation device or the laryngoscope blade so that the first view screen and the second view screen are viewable together by the operator during use. The stylet can be curveable at its distal end adjacent the patient's airway passage by a translating means mounted on the operator handle (e.g., when present) and connected to the distal end of the stylet to curve the distal end of the stylet, for example with a handle-mounted trigger for squeezing by one hand of the operator to actuate the translating means and curve the stylet distal end while the operator's other hand manipulates the laryngoscope blade. The laryngoscope blade can be curved to depress the patient's epiglottis with one side of the laryngoscope blade

The disclosure also relates a method for placing an endotracheal tube in a patient by an operator, the method comprising: (a) providing the endotracheal intubation system in any of its various disclosed embodiments, the endotracheal intubation system further comprising an endotracheal tube mounted to the stylet; (b) inserting the laryngoscope blade into the patient's airway passage and manipulating the laryngoscope blade controlled by one hand of the operator to lift the patient's epiglottis while viewing the second image of the patient's airway passage during insertion and manipulation of the laryngoscope blade; and (c) placing the endotracheal tube in the patient's airway passage with the endotracheal intubation device controlled by another hand of the operator while viewing the first image of the patient's airway passage during placement of the endotracheal tube. In a refinement of the method, (i) inserting and manipulating the laryngoscope blade into the patient's airway passage defines an open passageway between a surface of the laryngoscope blade and surrounding soft tissue in the patient's airway passage; and (ii) placing the endotracheal tube in the patient's airway passage comprises inserting the endotracheal tube through the open passageway and into the to the patient's trachea. In another refinement, placing the endotracheal tube in the patient's airway passage comprises advancing at least a distal portion of the endotracheal tube beyond the distal end of the laryngoscope blade and within a field of view of the second camera means while viewing the second image of the endotracheal tube during advancement of the endotracheal tube. Suitably, placing the endotracheal tube in the patient's airway passage can comprise advancing the endotracheal tube into the patient's trachea while viewing the second image of the endotracheal tube during advancement of the endotracheal tube to control a degree of insertion of the endotracheal tube into the trachea. For example, (i) the endotracheal tube can comprise externally visible length-indicating means thereon, and (ii) controlling the degree of insertion of the endotracheal tube can comprise viewing the second image of the length-indicating means during advancement of the endotracheal tube into the trachea. More specifically, (i) the endotracheal tube can comprise externally visible length-indicating means thereon, (ii) inserting the laryngoscope blade into the patient's airway passage can comprise advancing the blade to a position where the patient's vocal cords are at least partially within the field of view of the second camera means, and (iii) controlling the degree of insertion of the endotracheal tube can comprise viewing the second image of the length-indicating means at a position above the vocal cords during advancement of the endotracheal tube into the trachea to attain a preselected linear insertion distance of the distal end of the endotracheal tube into the trachea and relative to the vocal cords (e.g., at least about 2 cm, ranging between about 3 cm and about 5 cm).

The present disclosure relates to an endotracheal intubation system adapted for two handed operation by a medical operator, which comprises: (a) an endotracheal intubation device with a stylet adapted for mounting an endotracheal tube, which stylet is mounted at a proximal end on a handle for the operator and connected to a monitor or monitors, wherein the system is adapted to simultaneously receive visual images from at least two different digital camera means, a first camera means of which is mounted adjacent a distal end of the stylet and enabling viewing of the airway passage in a patient on a first screen; and (b) a laryngoscope blade, for lifting the epiglottis of the patient simultaneously with the viewing of the airway passage by the intubation device, with a second camera means which enables viewing of a second image on a second screen adjacent to the first screen, wherein in use, the intubation device and blade at a proximal end of each is adapted to be manipulated together by either the left or the right hands of the operator together. Further, the present disclosure relates to a system wherein a stylet for holding the endotracheal tube is curveable at a distal end adjacent the airway by a translating means mounted on the handle and connected to the distal end of the stylet to curve the distal end. The camera means is preferably a CMOS or CCD device. Preferably, the blade is curved to depress the epiglottis with one side of the blade. Still preferably, the first and second screens are mounted on a pole for viewing by the operator simultaneously. In another embodiment, the screen is mounted on the intubation device on the handle so that the first and second screens are viewable together by the operator. Further, the system is preferably adapted to enable the operator to squeeze a trigger on the handle with one hand while manipulating the blade with the other hand.

The present disclosure relates to the placement of an endotracheal tube by a medical operator which comprises: (a) providing an endotracheal intubation device with a stylet adapted for mounting an endotracheal tube, which stylet is mounted at a proximal end on a handle for the operator and connected to a monitor or monitors, wherein the system is adapted to simultaneously receive visual images from at least two different digital camera means, a first camera means of which is mounted adjacent a distal end of the stylet and enabling viewing of the airway passage in a patient on a first screen; and a laryngoscope blade, for lifting the epiglottis of the patient simultaneously with the viewing of the airway passage by the intubation device, with a second camera means which enables viewing of a second image on a second screen adjacent the first screen, wherein in use, the intubation device and blade at a proximal end of each is adapted to be manipulated together by either the left or the right hands of the operator together; and (b) placing the endotracheal tube in the patient in the airway of the patient with the endotracheal tube device and manipulating the blade to lift the epiglottis while viewing the screen. Preferably, the stylet for holding the endotracheal tube is curved at a distal end adjacent the airway by a translating means mounted on the handle and connected to the distal end of the stylet to curve the distal end. Preferably, the camera means is a CMOS or CCD device. Preferably, the blade is curved to depress the epiglottis with one side of the blade. Preferably, the first and second screens are mounted on a pole for viewing by the operator simultaneously. In another embodiment, the screens are mounted on the intubation device on the handle so that the first and second screens are viewable by the operator simultaneously. Preferably, the operator squeezes a trigger with one hand while manipulating the blade with the other hand.

### BRIEF DESCRIPTION OF THE DRAWINGS

FIG. 1 is a front view showing the use of an intubation system including an endoscopic device 10 and a laryngoscope blade 400 together with a split view video monitor/display means 323A which allows viewing of the airway of the patient P with both devices by the medical operator M.

FIG. 2 is a front view of the monitor 323A.

FIG. 3 shows a front view of the blade 400 as shown in FIG. 1.

FIG. 4 is a distal end view of the blade 400 of FIG. 3 at line 4-4.

FIG. 5 is a cross-sectional view of the blade 400 of FIG. 3 at line 5-5.

FIG. 6 is a front cross-sectional view of a human head showing the positioning of the endotracheal tube 12 in the airway using the blade 400 and the endoscopic device 10.

FIG. 7 is a front view of a laryngoscope blade 420 with a wireless system mounted in the proximal end.

FIG. 8 is a proximal end view of the blade 420 along line 8-8.

FIG. 9 is a front view of an intubation system analogous to that of FIG. 1 where a screen/display means 501 is mounted on a pole 500, rather than on the endoscopic device 600.

FIG. A.1 illustrates an exemplary endotracheal intubation device with a trigger and hand grip in use on a patient by a medical professional.

FIG. A.2 illustrates a side view of the device of FIG. A.1.

FIGS. A.3A-A.3B illustrate an exemplary detachable stylet.

FIG. A.4A-A.4E illustrate various side views of the device from FIG. A.1 showing the display means in different orientations and an exemplary camera mounted in the tip.

FIG. A.5 illustrates a close-up view of the detachable stylet mounted in the hand grip of FIGS. A.3A and A.3B.

FIG. A.6 illustrates the opposite side view of FIG. A.5 of the hand grip having the detachable stylet mounted thereto.

FIG. A.7 illustrates a trigger side view of the device of FIG. A.1.

FIG. A.8 illustrates a side view with the grip housing removed from the device of FIG. A.1.

FIG. A.9 illustrates an opposite side view of FIG. A.8 with the internal components exposed showing the actuator assembly.

FIG. A.10 illustrates a close-up view of the actuator assembly in connection with the trigger mechanism.

FIG. A.11 illustrates then internal components of the device of FIG. A.1 with the retaining plate.

FIG. A.12 illustrates the actuator assembly from the side of the stylet engaged with the trigger mechanism.

FIG. A.13 illustrates a close-up view of the actuator assembly of FIG. A.12 with the trigger removed.

FIG. A.14 illustrates the view of FIG. A.13 with the control wire and elongated tube removed.

FIG. A.15 illustrates a trigger side view of the actuator assembly engaged with the trigger mechanism with the grip housing removed.

FIG. A.16 illustrates a trigger side view of FIG. A.15 with the actuator housing removed.

FIG. A.17 illustrates a trigger side view of FIG. A.16 with the linkage removed.

FIG. A.18 illustrates an exemplary display means having a monitor and power supply.

FIG. A.19 illustrates the display means of FIG. A.18 illustrating internal components.

FIG. A.20 illustrates an exemplary embodiment of an endotracheal intubation device having a hand grip with display means and trigger engaged with a stylet assembly.

FIG. A.21 illustrates the device of FIG. A.20 with the display means in a rotated and adjusted orientation.

FIG. A.22 illustrates the device of FIG. A.21 with the display means in a rotated, adjusted and swiveled orientation.

FIGS. A.23A-A.23D illustrate an exemplary endotracheal intubation device having an exemplary hand grip with a grooved trigger and a display monitor.

FIG. A.24A illustrates a perspective view of an exemplary detachable stylet.

FIG. A.24B illustrates a side view of the stylet of FIG. A.24A showing the internal components of the actuator assembly.

FIG. A.24C illustrates a further side view of the stylet from FIG. A.24A rotated 90° from the view of FIG. A.24B.

FIG. A.24D is a cross section view A-A from FIG. A.24B showing an exemplary articulation section.

FIG. A.24E is a cross section view B-B from FIG. A.24C showing an exemplary soak cap mounted on the electrical connection.

FIG. A.24F is a cross section view C-C from FIG. A.24B showing an actuating mechanism inside the actuator housing.

FIG. A.24G illustrates a distal tip view of the stylet of FIG. A.24A.

FIG. A.25A illustrates a perspective view of an exemplary grip housing with a monitor and trigger.

FIG. A.25B illustrates a trigger side view of the grip housing of FIG. A.25A.

FIG. A.25C illustrates a side view showing the mounting and pivoting of the monitor to the grip housing of FIG. A.25A.

FIG. A.26A is a cross section view A-A of FIG. A.25B.

FIG. A.26B is a cross section view B-B of FIG. A.26A.

FIG. A.26C is a cross section view C-C of FIG. A.26A.

FIG. A.26D is a cross section view D-D of FIG. A.25C.

FIG. A.27A illustrates a perspective view of an exemplary grip housing without the trigger connected.

FIG. A.27B illustrates an exemplary trigger with finger grooves.

FIG. A.28A-A.28C illustrate an exemplary linkage with a single opening shown in perspective (A) view, backbone side down (B) view, and backbone side up (C) view.

### DETAILED DESCRIPTION

All patents, patent applications, government publications, government regulations, and literature references cited in this specification are hereby incorporated herein by reference in their entirety. In case of conflict, the present description, including definitions, will control.

Additional features of the disclosure may become apparent to those skilled in the art from a review of the following detailed description, taken in conjunction with the examples, drawings, and appended claims, with the understanding that the disclosure is intended to be illustrative, and is not intended to limit the claims to the specific embodiments described and illustrated herein.

The disclosed dual-screen intubation system is an improvement over endoscopic/endotracheal intubation devices such as those disclosed in U.S. Publication No. 2010/0095969 to Schwartz et al. and incorporated herein by reference in its entirety. The improvement herein is in the use of a laryngoscope blade with a camera at the distal end of the laryngoscope blade in addition to a camera at the distal end of the endoscopic device, thus enabling viewing of a patient's airway passage from the tip of the blade as well as the tip of intubation device. The view of the airway passage from the tip of the intubation device and from the tip of the blade is as seen on first and second screens of an image/video display. Thus, one view is of the image from the blade camera and the other view is for the image from the endoscopic device. The intubation system provides the physician performing an intubation procedure with both a view of the patient's airway (e.g., from the blade camera) and a simultaneous view of the intubation device during placement of the intubation tube. A first display view is provided by the camera in the laryngoscope blade, thus allowing the physician to visualize and open the patient airway as well as to provide an insertion pathway for the intubation device and endotracheal tube. A second display view is provided by the camera in the distal end of the endoscopic device (e.g., in the stylet thereof) that carries the endotracheal tube into the blade-opened airway and past the vocal chords, where the endotracheal tube can then be removed from the stylet to intubate the patient.

### Endotracheal Intubation System

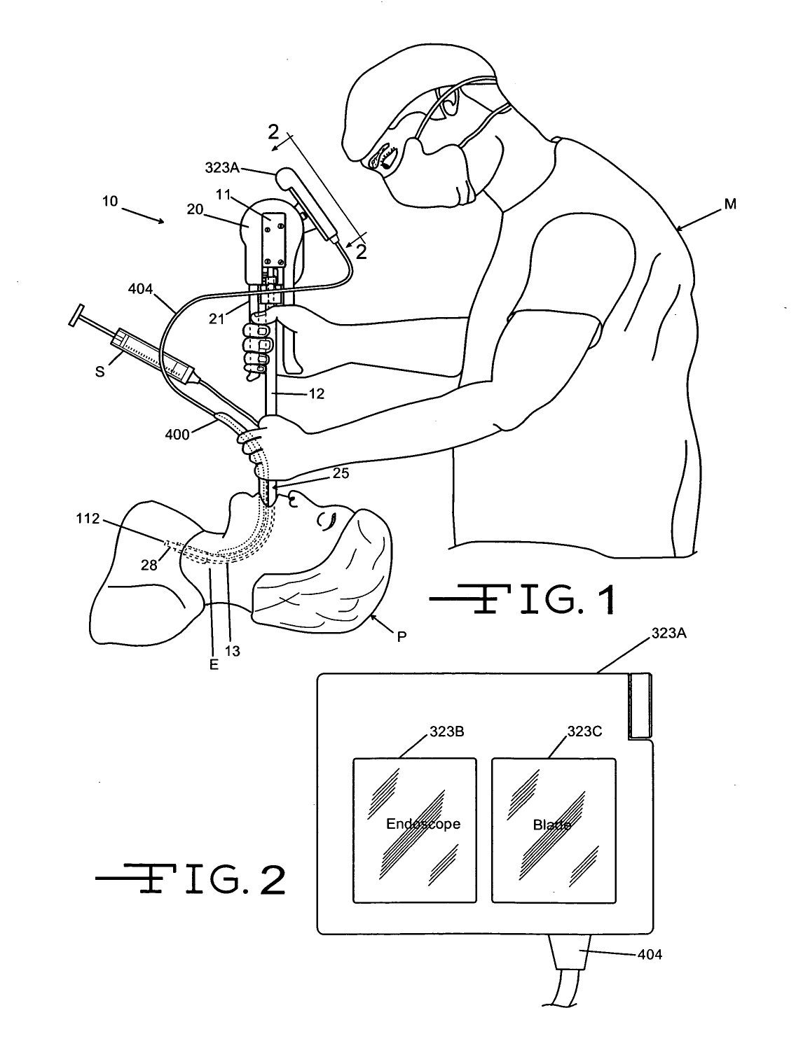

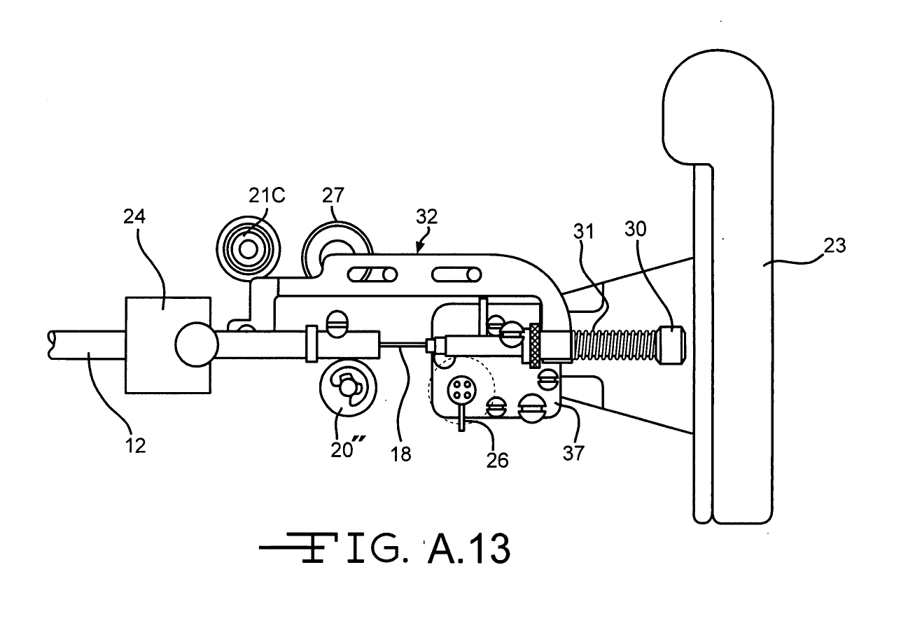

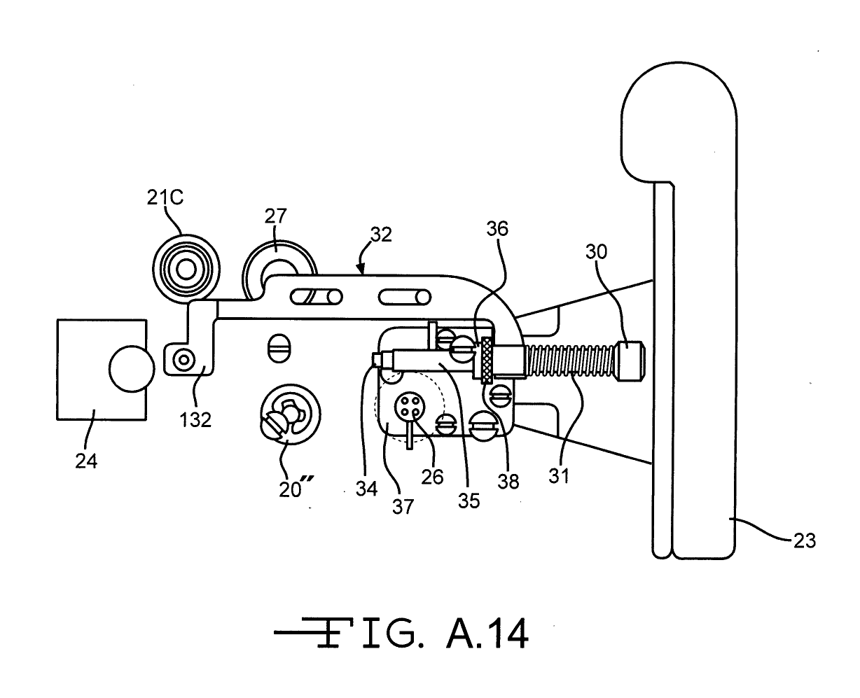

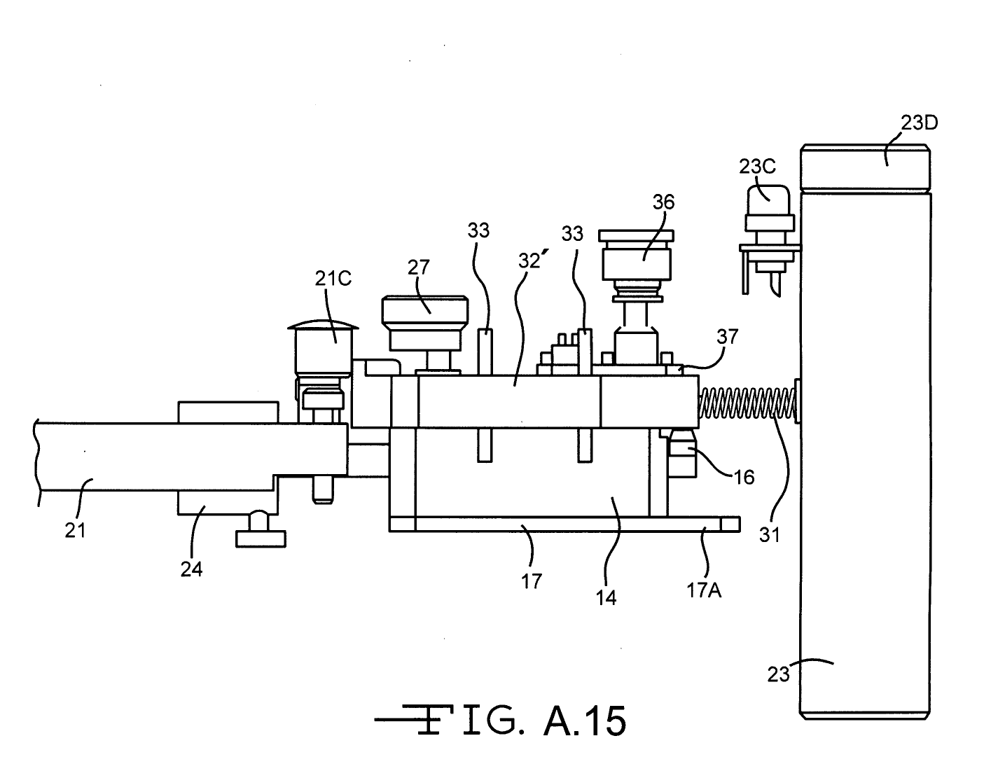

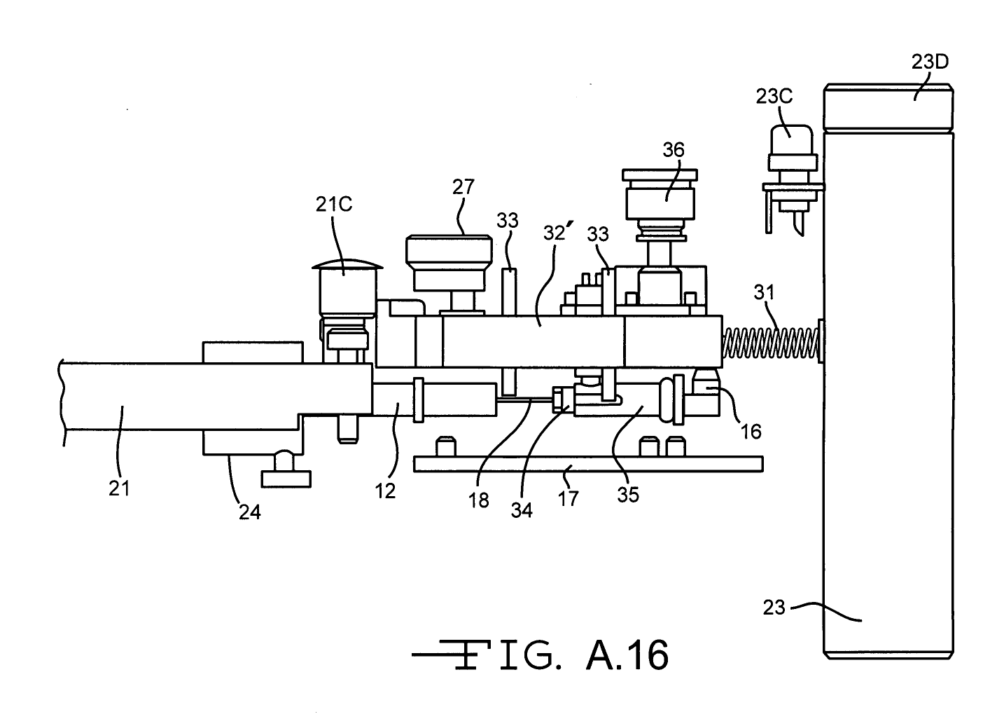

FIGS. 1-8 illustrate various embodiments of an endotracheal intubation system according to the disclosure as well as related methods of use. As generally shown in FIG. 1, a dual-screen intubation system includes an endotracheal intubation device (or endoscope) 10 for inserting an endotracheal tube 25 into a patient's airway passage while viewing the passage, a laryngoscope blade 400 for lifting a patient's epiglottis with simultaneous viewing of the patient's airway passage by the endotracheal intubation device, and a display means 323A (e.g., a monitor with one or more view screens) for simultaneously receiving and viewing visual images from at least two different digital camera means. The endotracheal intubation system is adapted for two-handed operation by a medical operator M such that during use by the medical operator M, a proximal end of the intubation device 10 and a proximal end of the laryngoscope blade 400 are each adapted to be manipulated together in two-handed operation by the operator's left and the right hands together. Specifically, the operator controls the intubation device 10 with one hand and controls the laryngoscope blade 400 with the other hand while viewing real-time images of the patient's airway electronically transmitted from the system components 10, 400 to the display means 323A.

The endotracheal intubation device 10 is not particularly limited and generally can include devices known in the art for mounting and inserting an endotracheal tube 25 into a patient's airway passage and trachea E. The intubation device 10 generally includes a stylet 12 (e.g., elongated tube) with a distal end/tip 112. A first camera means 28 is mounted to the stylet 12 adjacent the distal end 112 thereof (e.g., internally mounted at an open distal end 112 of the stylet 12) and enables viewing of a first image of a patient's airway passage on a first view screen 323B of the display means 323A. The first camera means 28 can be a CMOS camera device or a CCD camera device (e.g., being capable of capturing and delivering real-time images or video) and suitably includes a light means 19 (e.g., LED lights) to illuminate the area captured by the first camera means 28.

Various refinements of the endotracheal intubation device 10 are possible, for example as described in detail below and illustrated in FIGS. A.1-A.28. The intubation device 10 suitably includes an operator handle 20 mounted to the stylet 12 at a proximal end thereof (e.g., detachably mounted to the handle 20 at a position 11 shown in FIG. 1). The stylet 12 can be curveable at the distal end 112 thereof (e.g., in an articulation section 13 adjacent the patient's airway passage when inserted therein in an intubation procedure) by a translating means mounted on the operator handle 20 and connected to the stylet distal end 112 to curve the stylet distal end 112. In such case, the handle 20 of the endotracheal intubation device 10 can include a trigger 21 for squeezing by one hand of the operator to actuate the translating means and to curve the stylet distal end 112 (e.g., along with the endotracheal tube 25 mounted thereto) while the operator's other hand manipulates the laryngoscope blade 400.

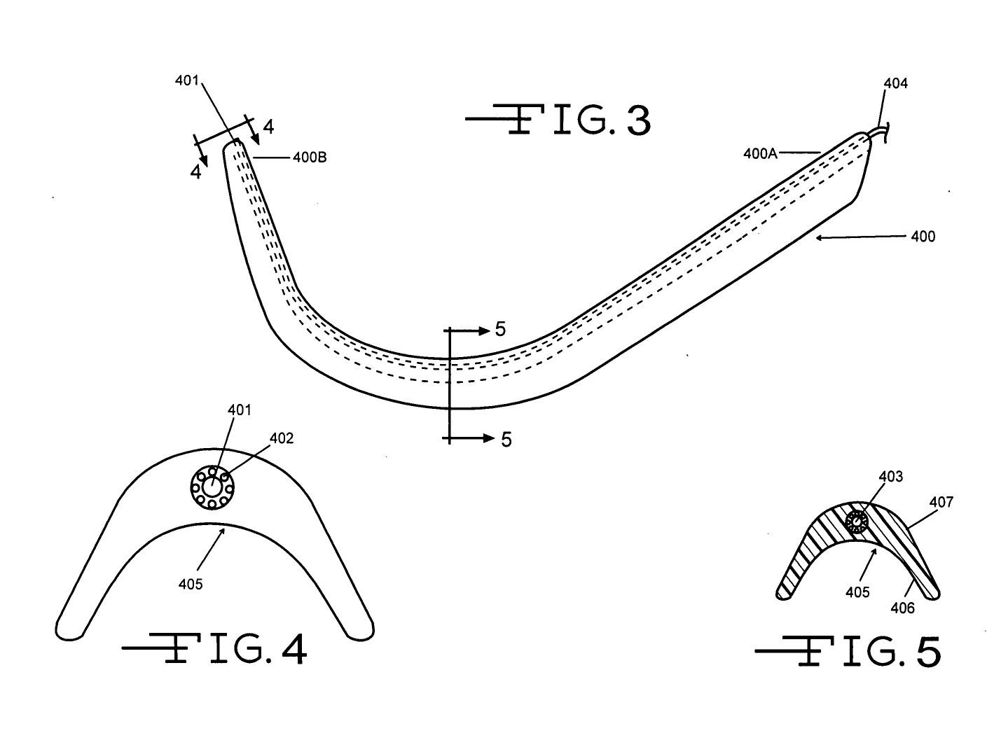

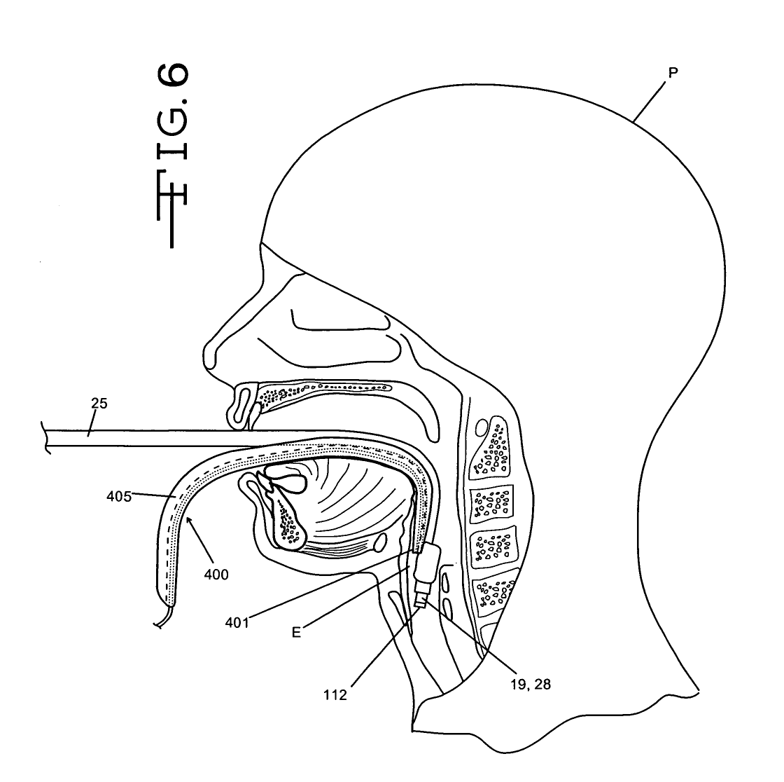

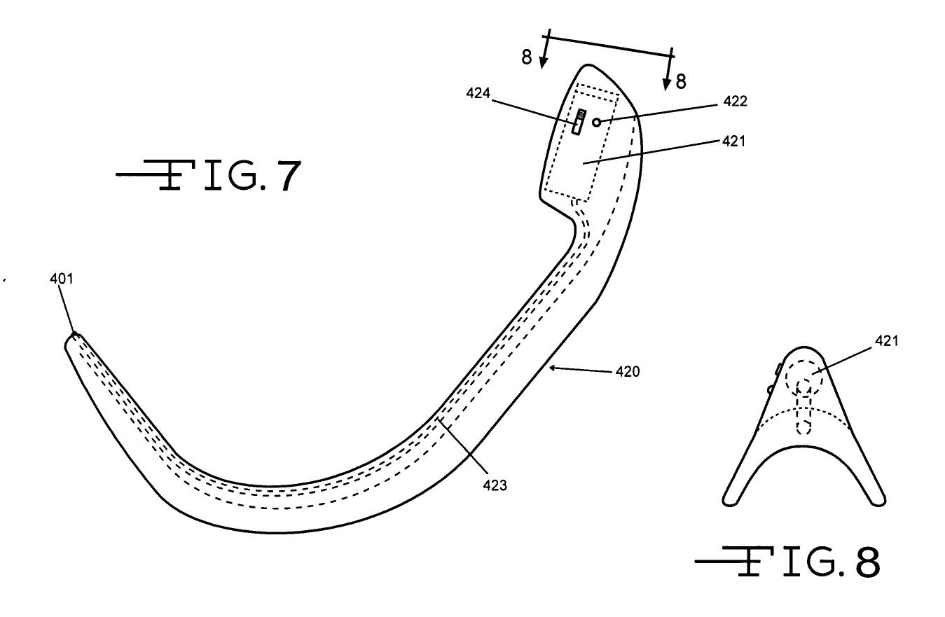

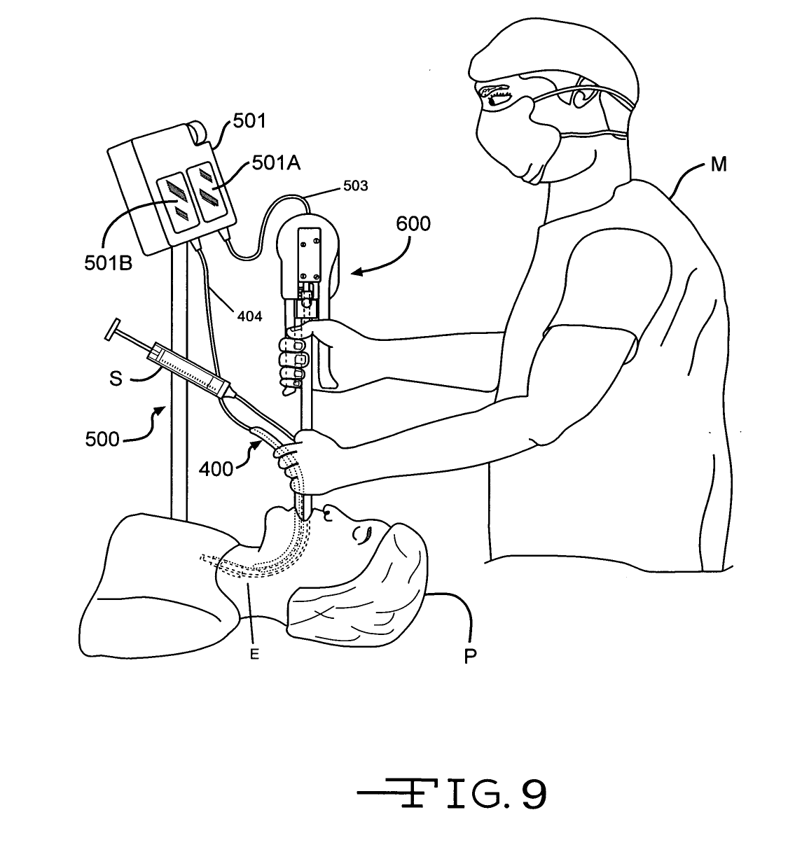

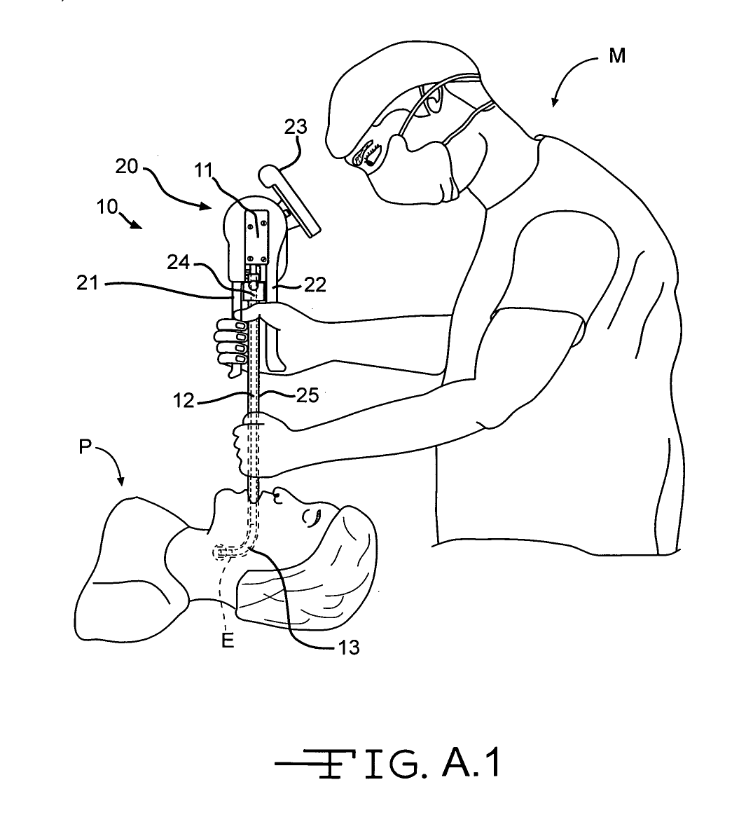

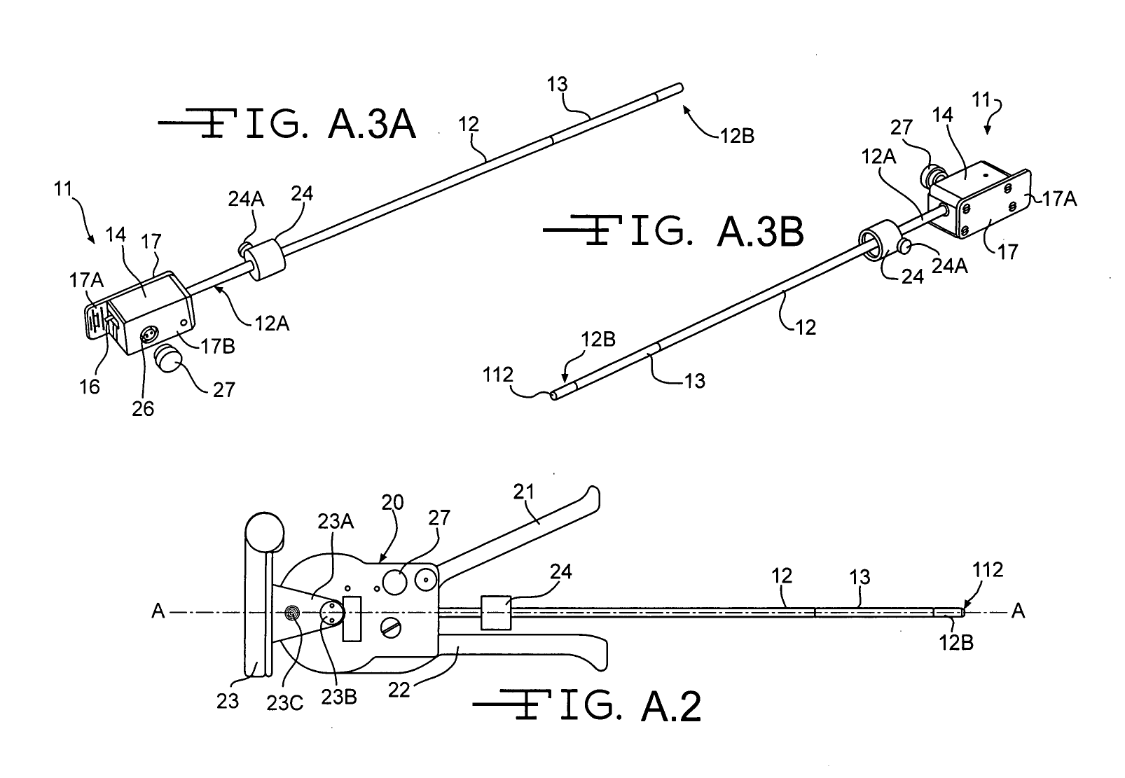

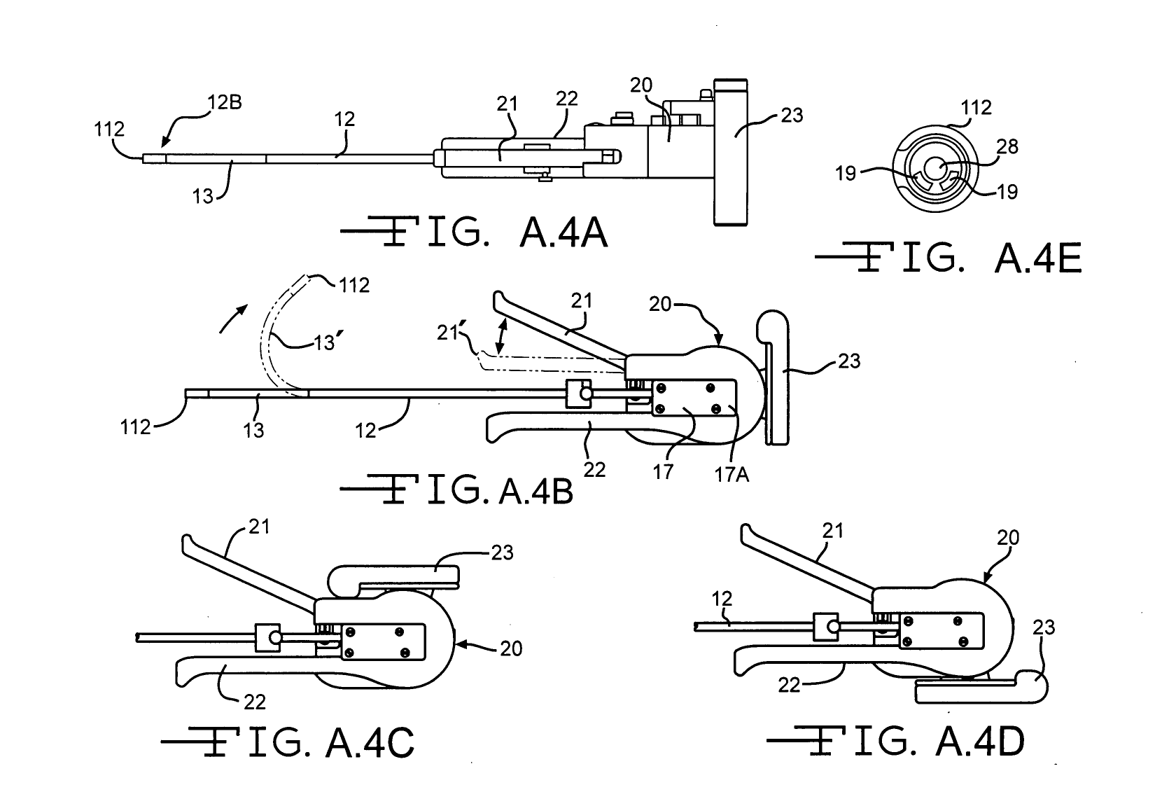

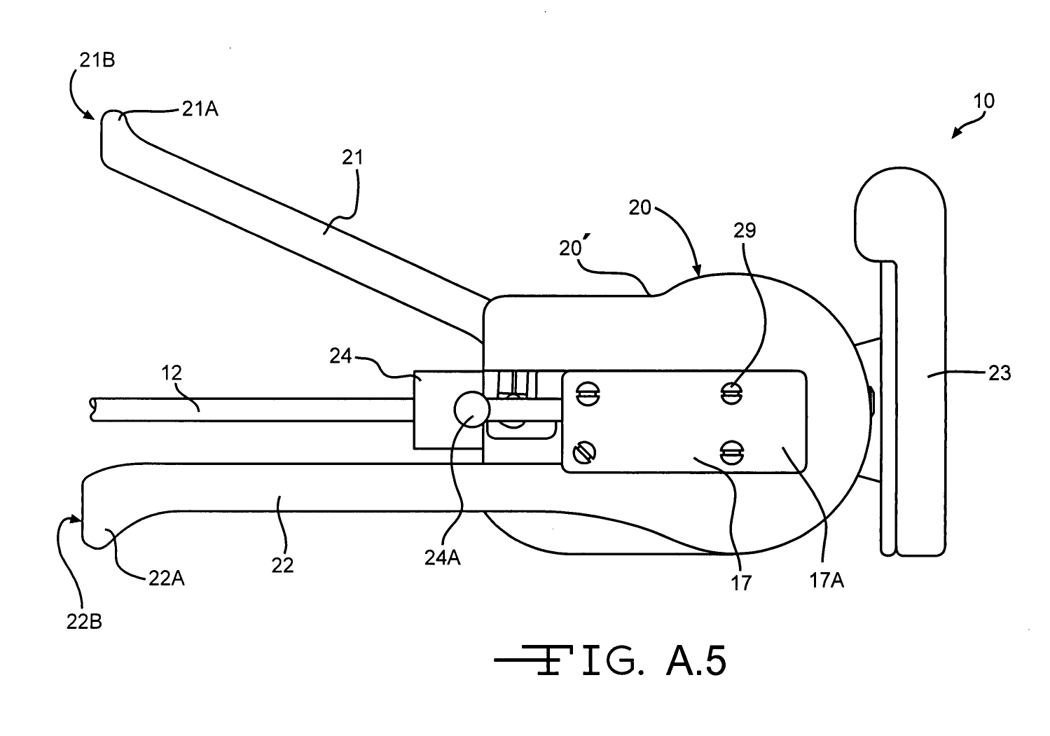

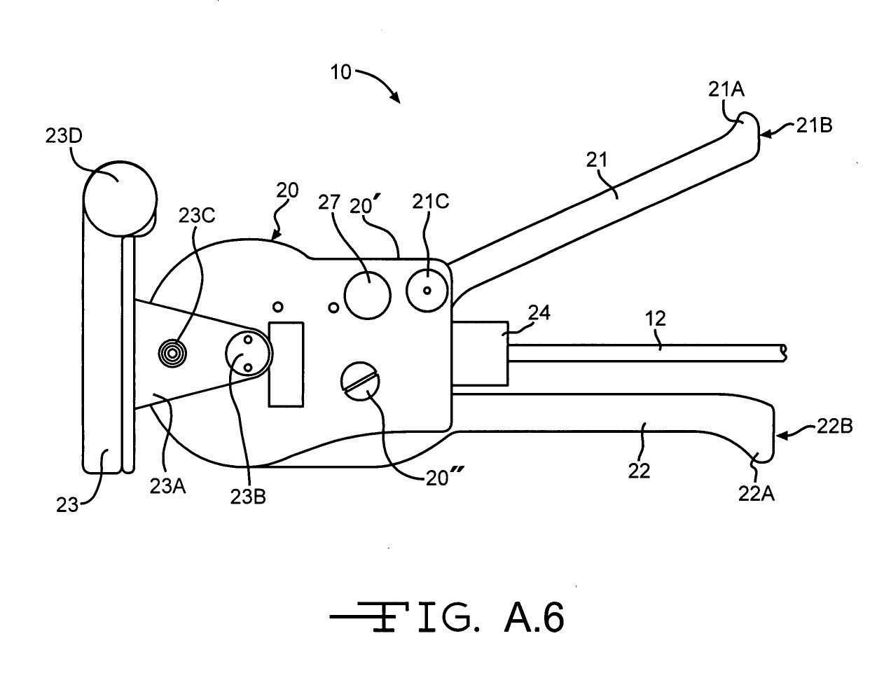

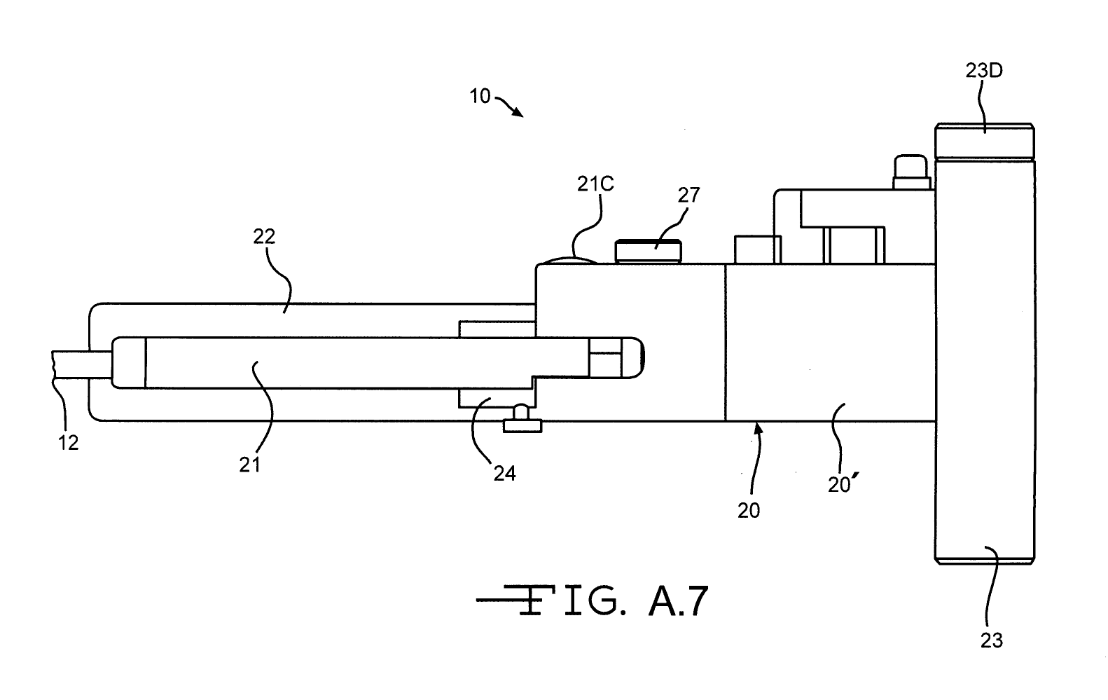

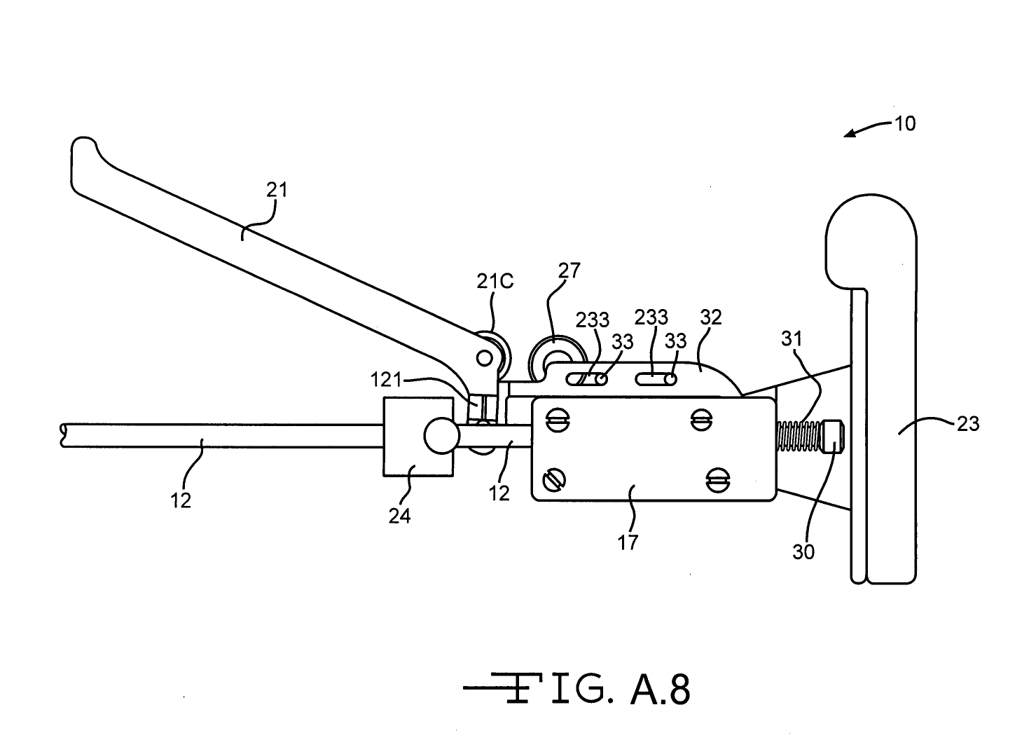

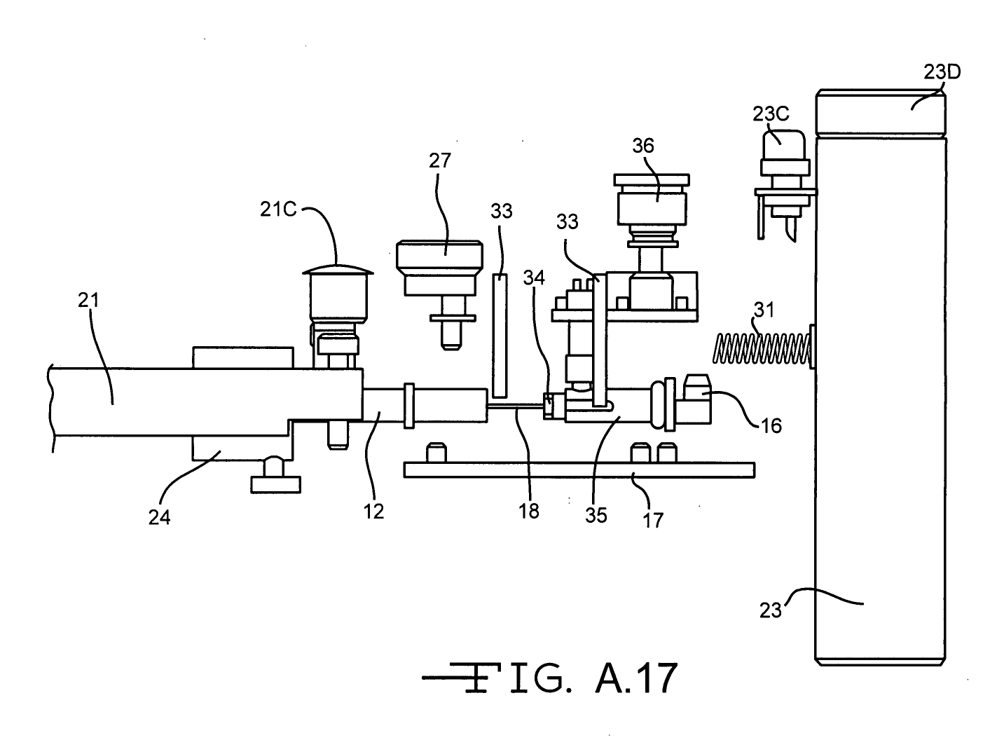

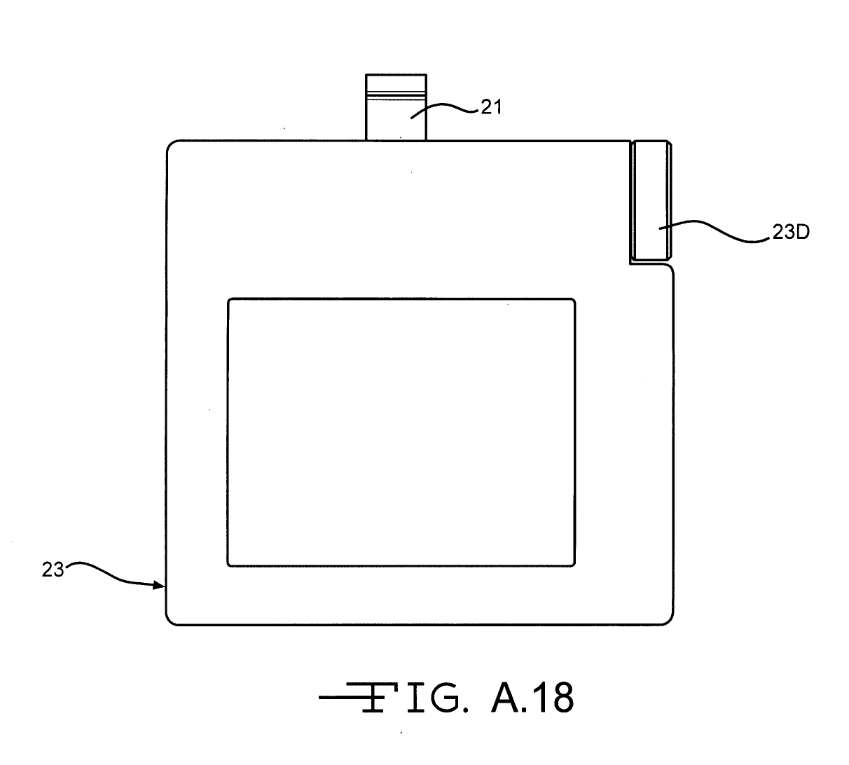

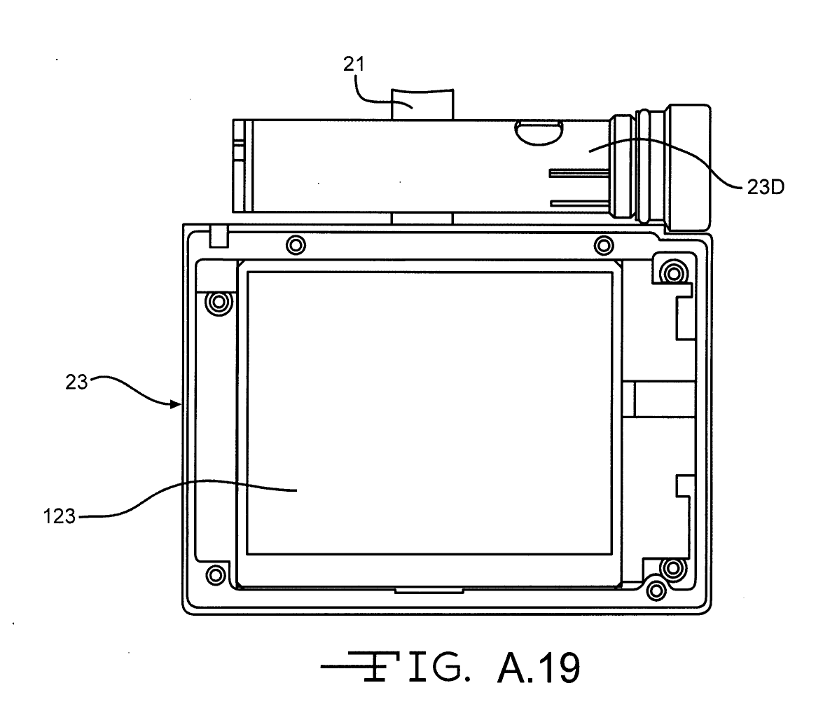

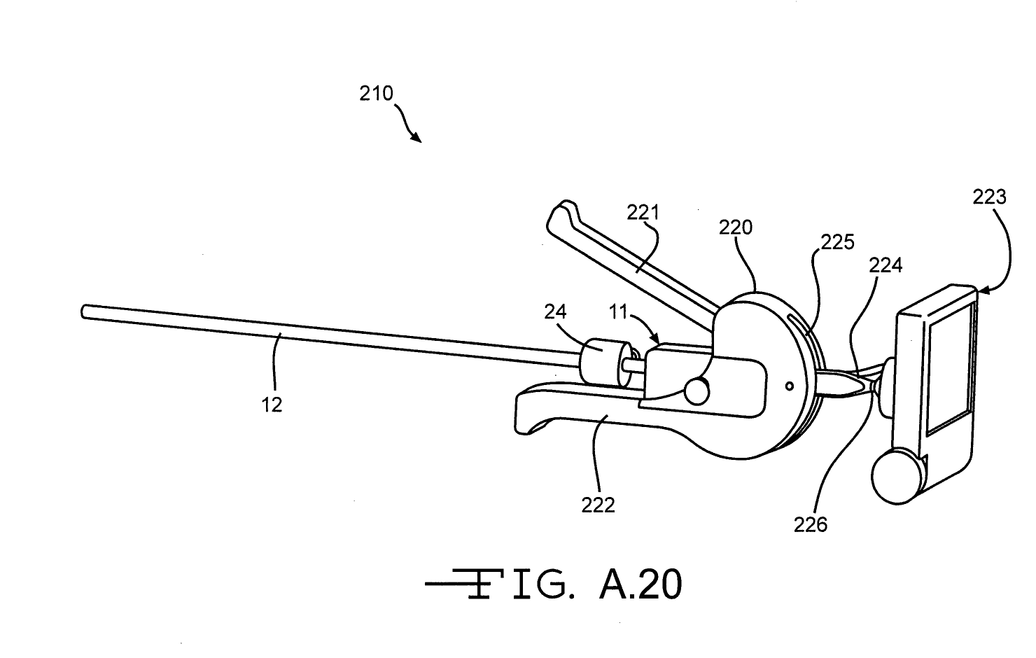

FIGS. 3-5 illustrate a suitable laryngoscope blade 400 according to the disclosure. The laryngoscope blade 400 has a proximal end 400A, has a distal end 400B, and includes a second camera means 401 mounted adjacent the blade distal end 400B. The second camera means 401 enables viewing of a second image of the patient's airway passage on a second view screen 323C of the display means 323A. The second camera means 401 can be a CMOS camera device or a CCD camera device (e.g., being capable of capturing and delivering real-time images or video) and suitably includes a light means 402 (e.g., LED lights) to illuminate the area captured by the second camera means 401. A cable 404 at the blade proximal end 400A is connected to the second camera means 401 via a wire 403 (e.g., internally mounted within blade 400 body) and provides a wired electronic connection to the display means 323A for transmission and display of the second image of the patient's airway. The laryngoscope blade 400 suitably includes a concave surface 406 defining a recess 405 that is adapted for positioning the endotracheal tube 25 mounted on the stylet 12 of the endoscope device 10 (i.e., is sized to accommodate the endotracheal tube 25 outer diameter) to follow the curvature of the blade 400 as shown in FIG. 6 during an intubation process. The recess 405 suitably has a parabolic geometry and forms a relatively smooth surface that has a substantially uniform cross-sectional shape extending along the curved length of the blade 400. The parabolic cross section of the recess 405 is open to the surrounding environment, and an endotracheal tube 25 following along smooth recess 405 surface is not enclosed by the blade 400. During insertion of the blade 400 into a patient's airway, an outer convex surface 407 of the blade 400 is typically in physical contact with at least the tongue and possibly the surrounding soft tissue of the patient P. This allows medical professional M to elevate the necessary tissues to allow for efficient and effective insertion of the endotracheal tube 25 into the trachea E (e.g., by lifting or depressing the epiglottis). For example, once the blade 400 is inserted, the blade 400 surface (e.g., surface 406/recess 405) and the surrounding soft tissue of the patient define an open passageway therebetween for endotracheal tube 25 insertion.

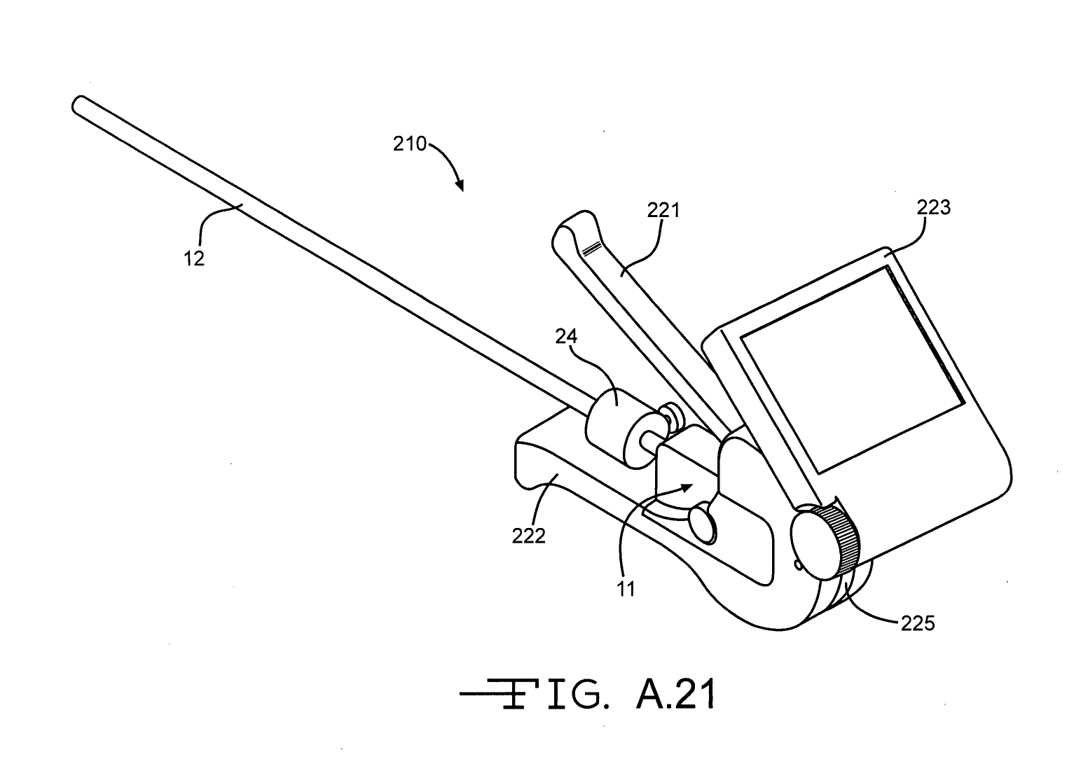

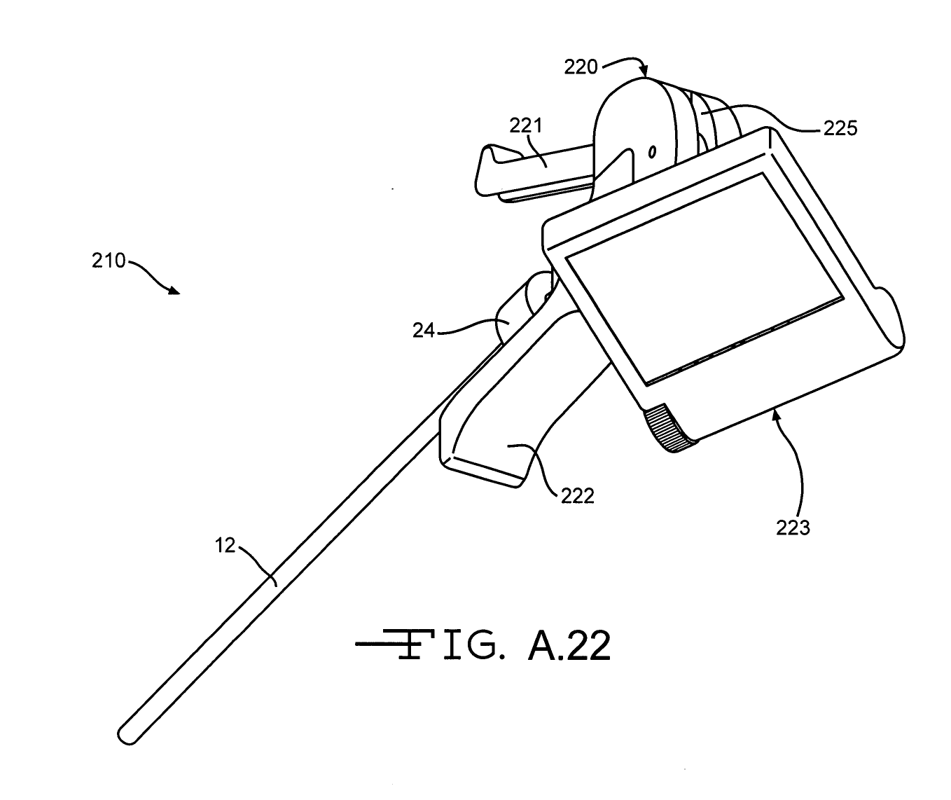

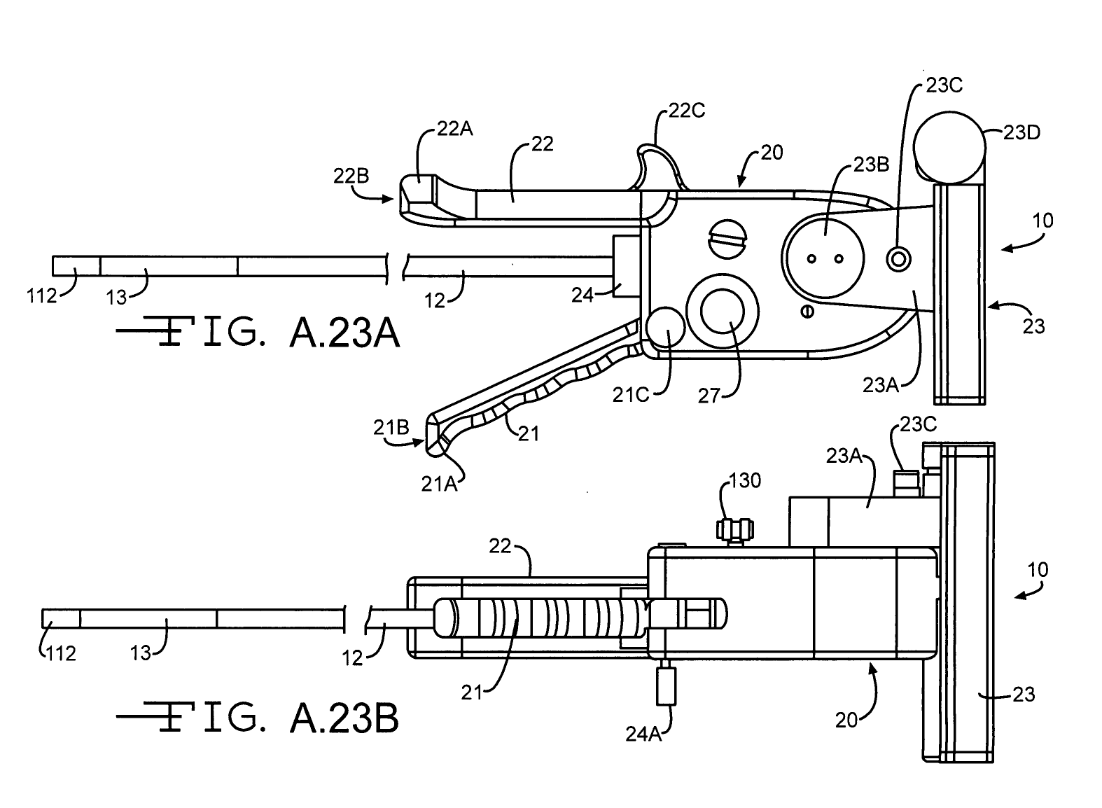

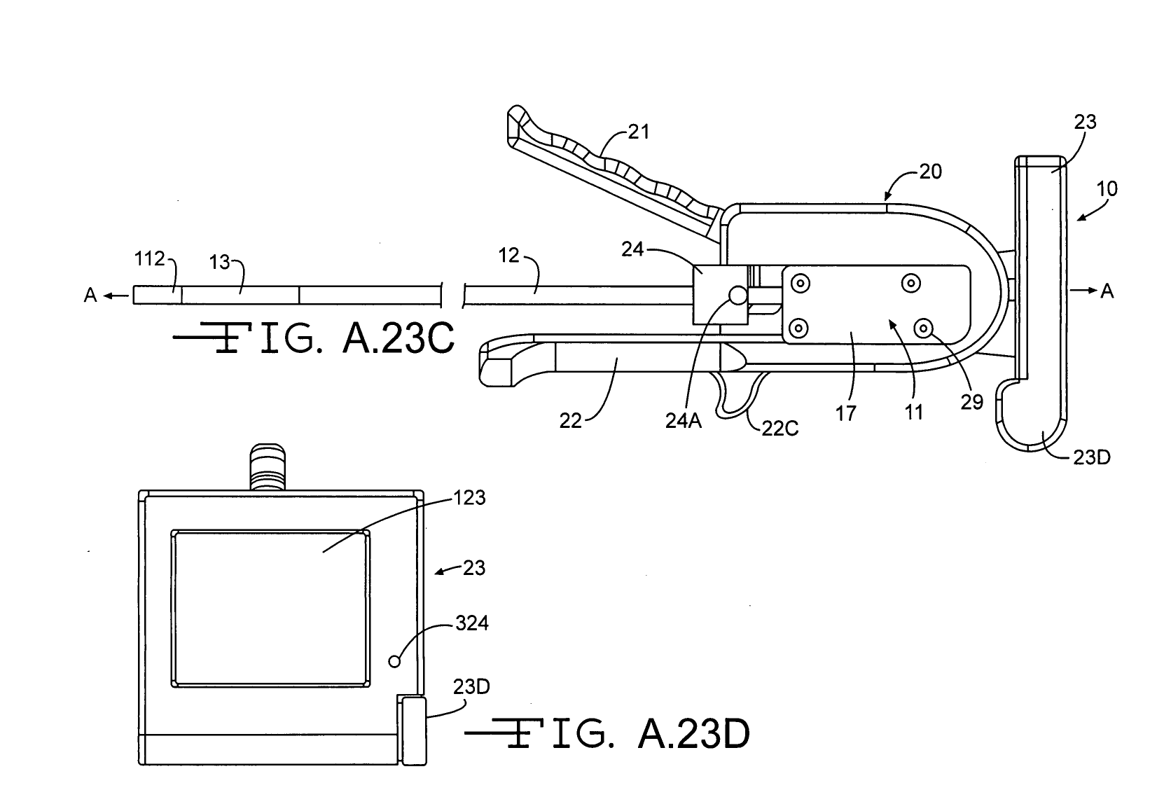

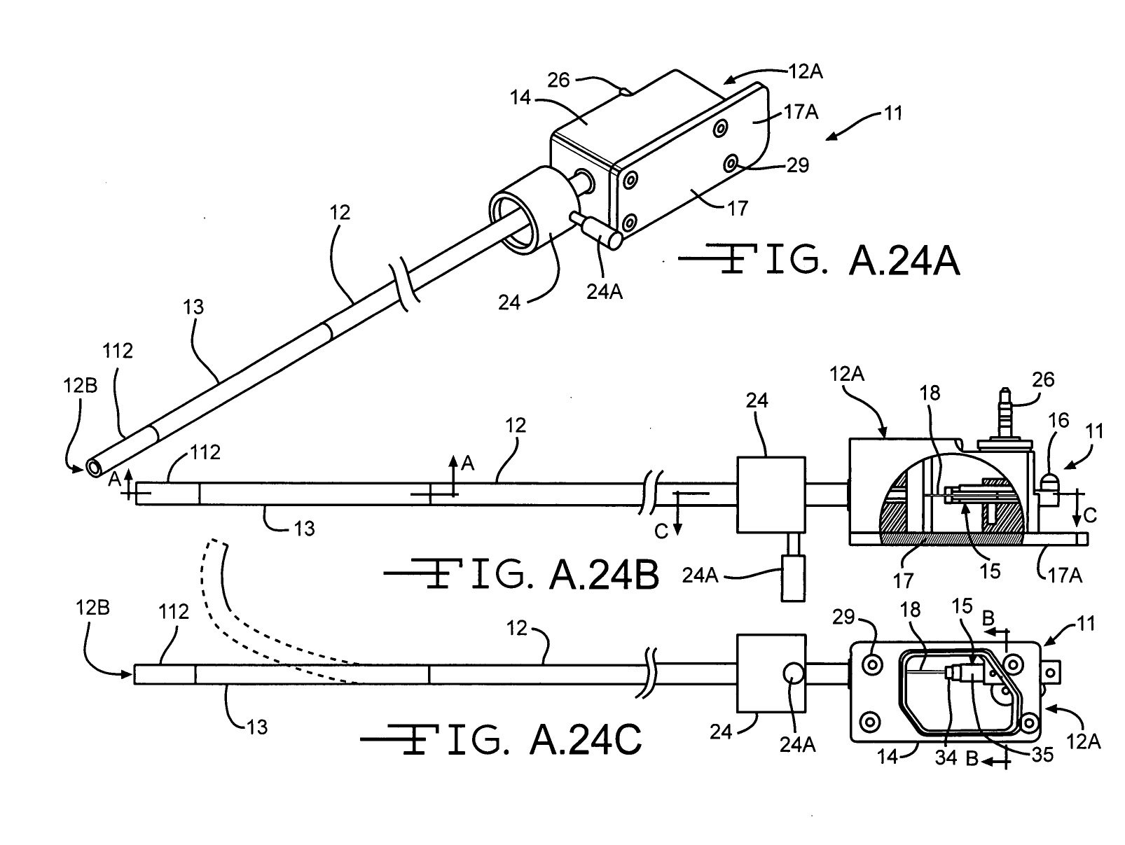

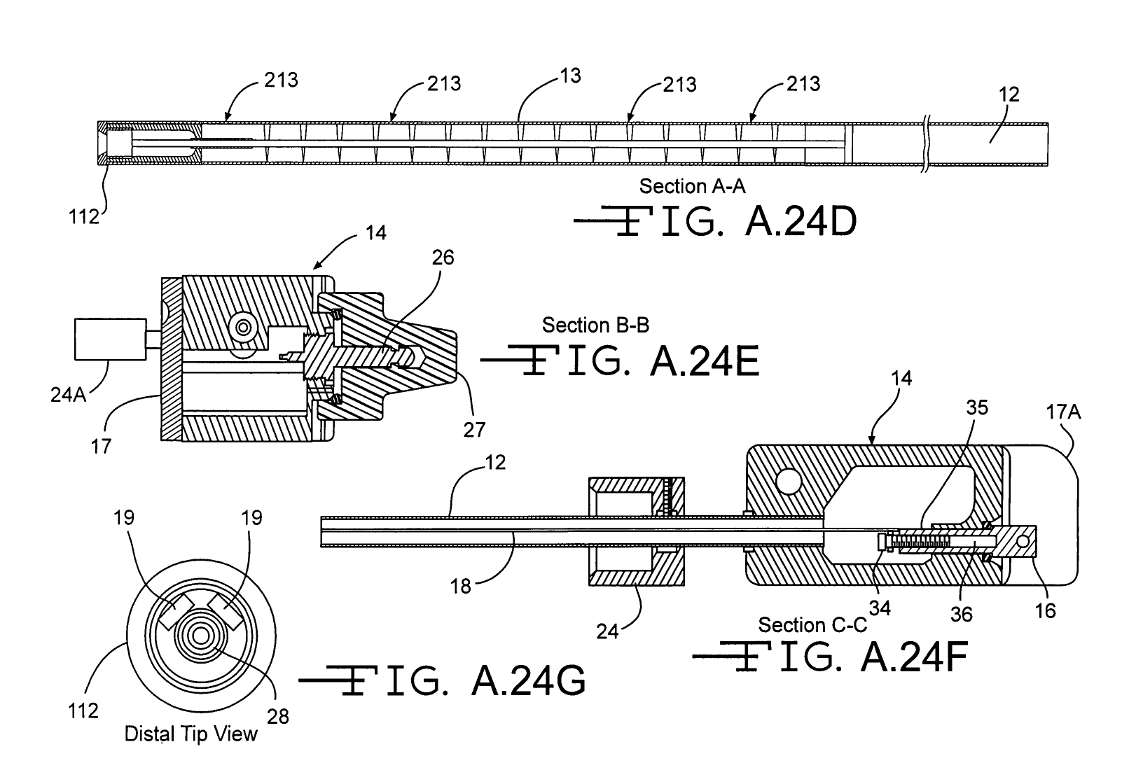

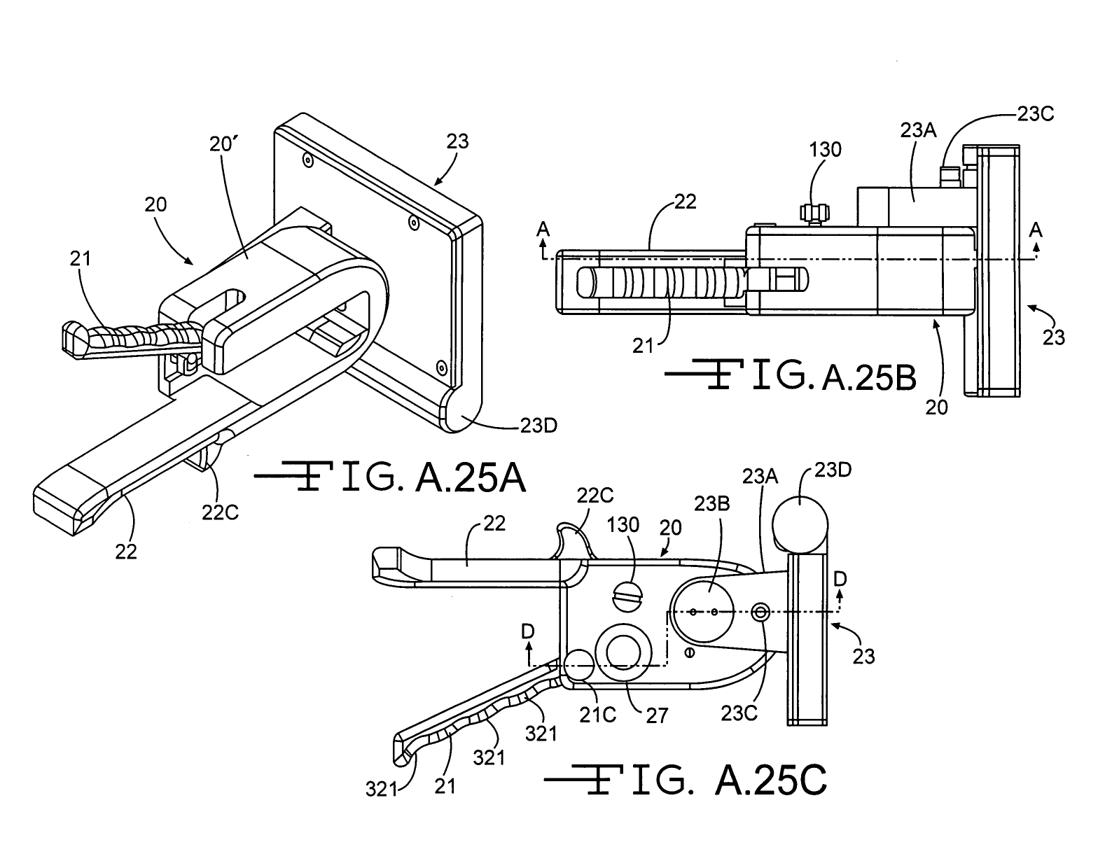

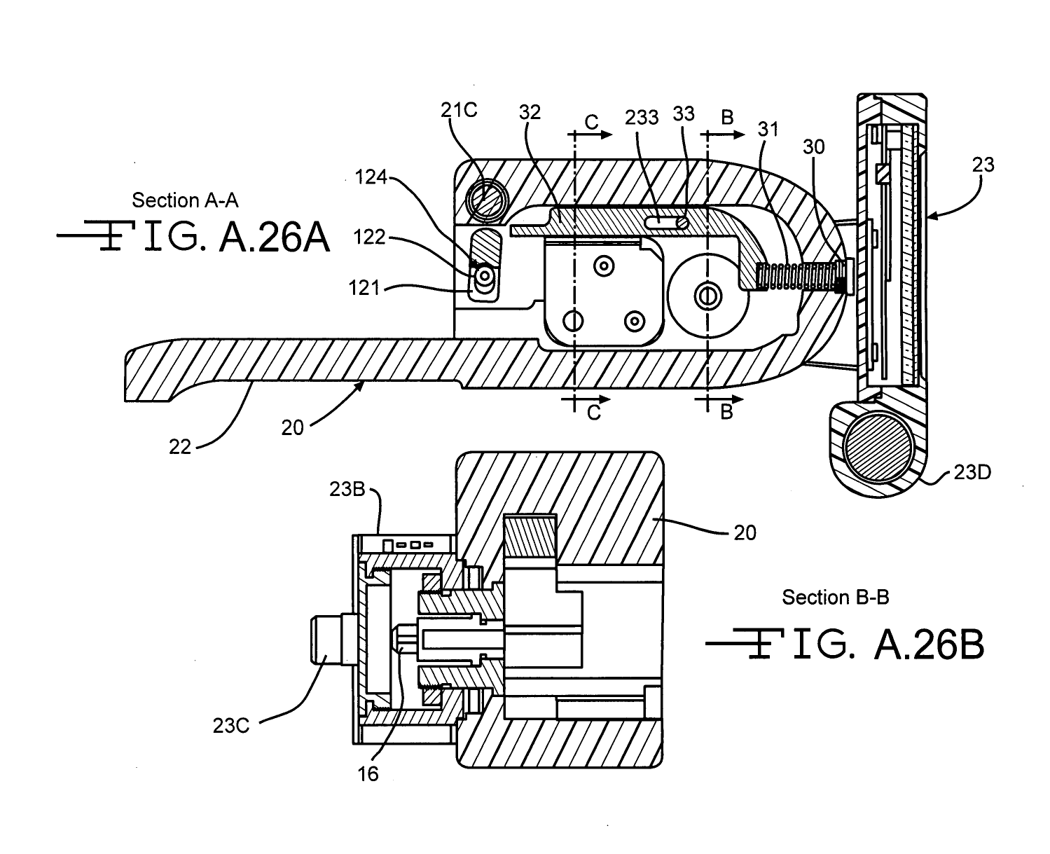

FIGS. 7-8 illustrate an alternate embodiment of a laryngoscope blade 420 which is constructed with an external structure analogous to that described above for the blade 400, but instead provides a wireless electronic connection between the second camera means 401 and the display means 323A. A wireless circuit 421 provides power (e.g., via a battery contained therein) to the second camera means 401 via an internally mounted wire 423 and provides a wireless communication link (e.g., with a radio transmitter/receiver contained therein) to the display means 323A when a switch 424 is turned on (e.g., as indicated by a light 422 such as a LED).

The display means 323A permits the simultaneous receiving and viewing of visual images (e.g., real-time video) from at least two different image input signals (e.g., separate digital inputs such as from separate digital camera means). Suitable electronic hardware for receiving and viewing visual images is known in the art. The display means 323A is electronically connected to the first camera means 28 and to the second camera means 401. The display means 323A includes a first view screen 323B for viewing the first image (i.e., from the intubation device 10) and a second view screen 323C for viewing the second image (i.e., from the blade 400). The first and second view screens 323B. 323C are positioned adjacent each other on the display means 323A (i.e., in close enough proximity so that the operator M can conveniently view both screens/images simultaneously).

The display means 323A includes one or more monitor screens (e.g., LCD display screens) than can independently and simultaneously display the first image and the second image from the first and second camera means 28, 401, respectively. For example, the display means 323A can include a single monitor screen 123 (e.g., as shown in FIG. A.23D) that provides simultaneous split-screen display and viewing (e.g., in a side-by-side or other arrangement) of the first and second images. Any desired spatial arrangement of the first and second images on the single physical monitor screen is possible, for example where the first view screen and the second view screen are virtual screens that occupy discrete regions of the physical monitor screen. In another embodiment and as shown in FIG. 2, the display means 323A is provided with a first monitor screen 323B and a second monitor screen 323C. Each monitor screen is a physically separate screen, where the first monitor screen 323B displays the first image and the second monitor screen 323C displays the second image (e.g., with each image occupying substantially the entire display area of its monitor screen).

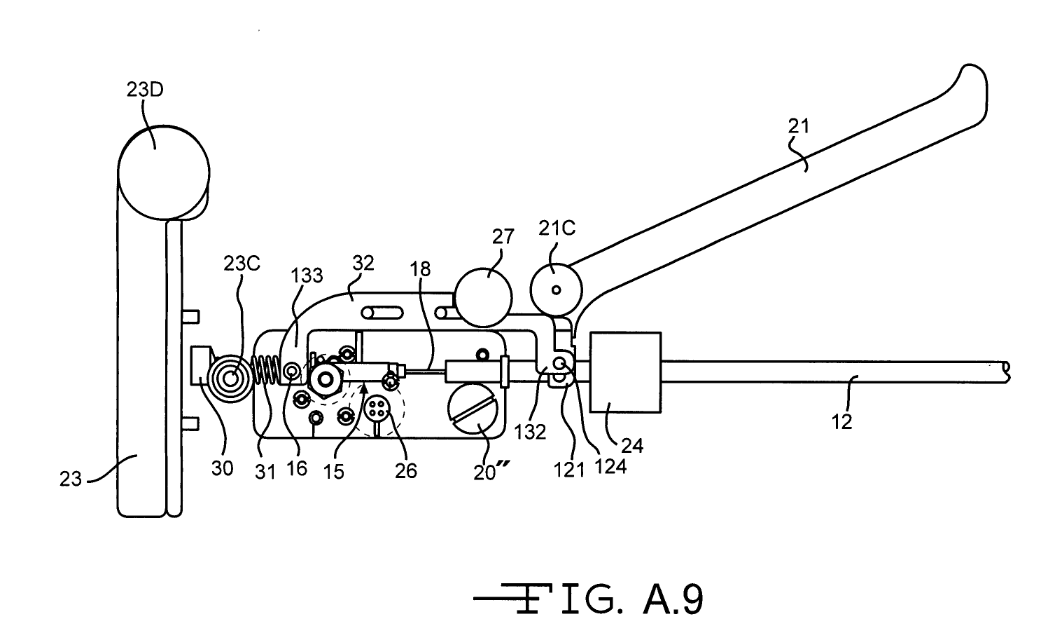

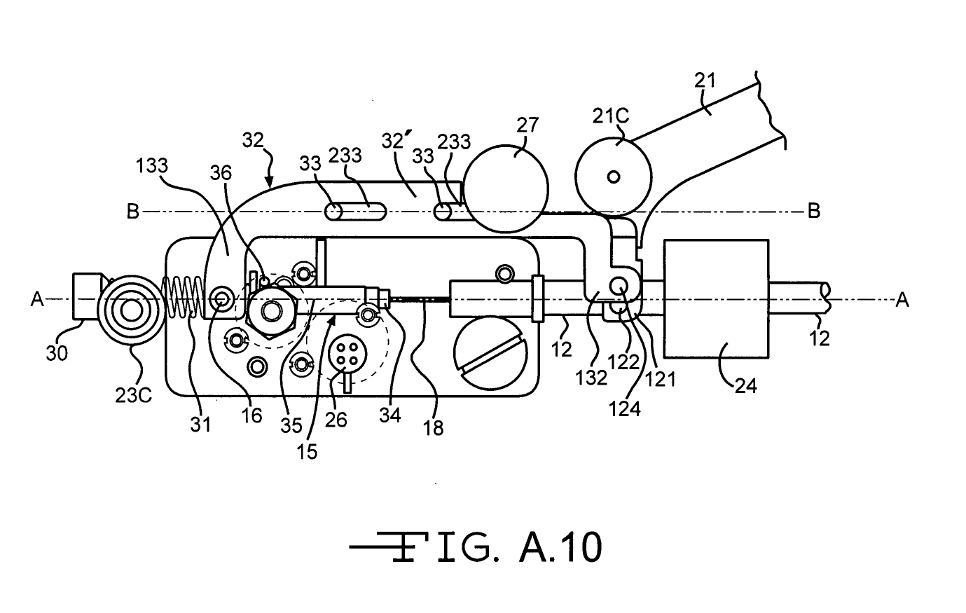

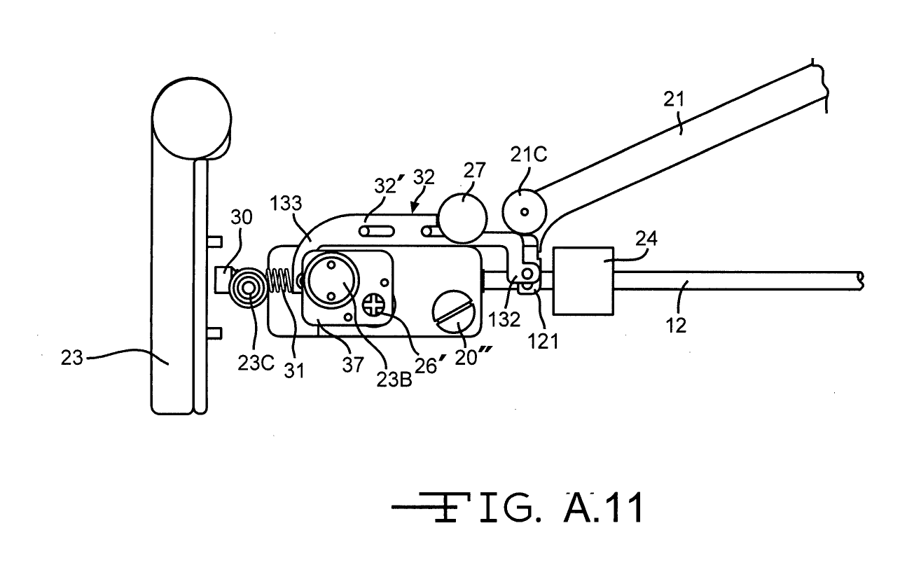

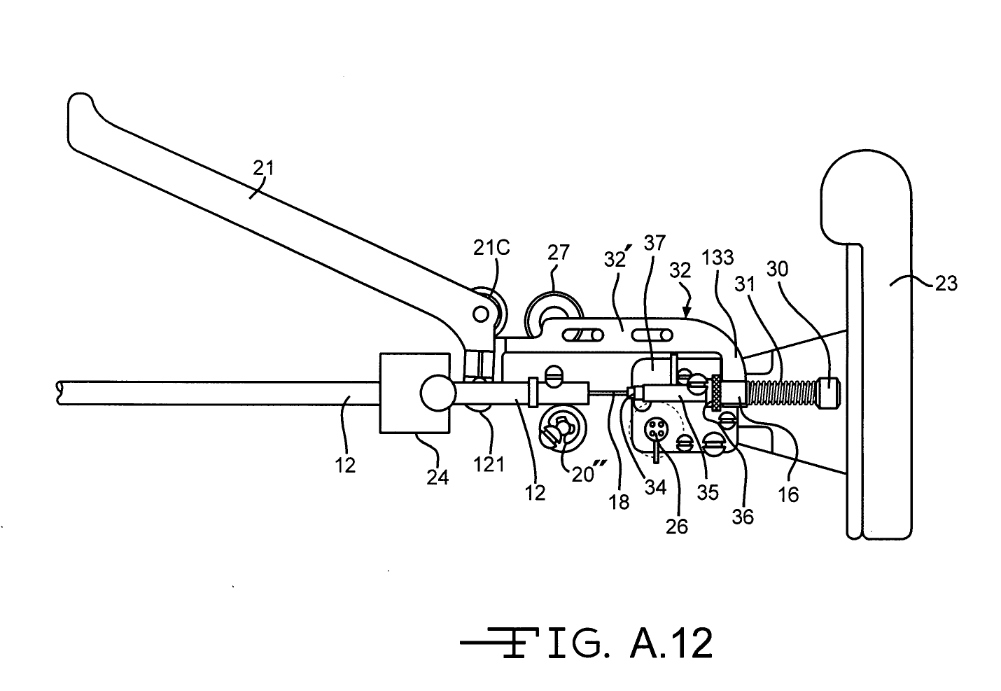

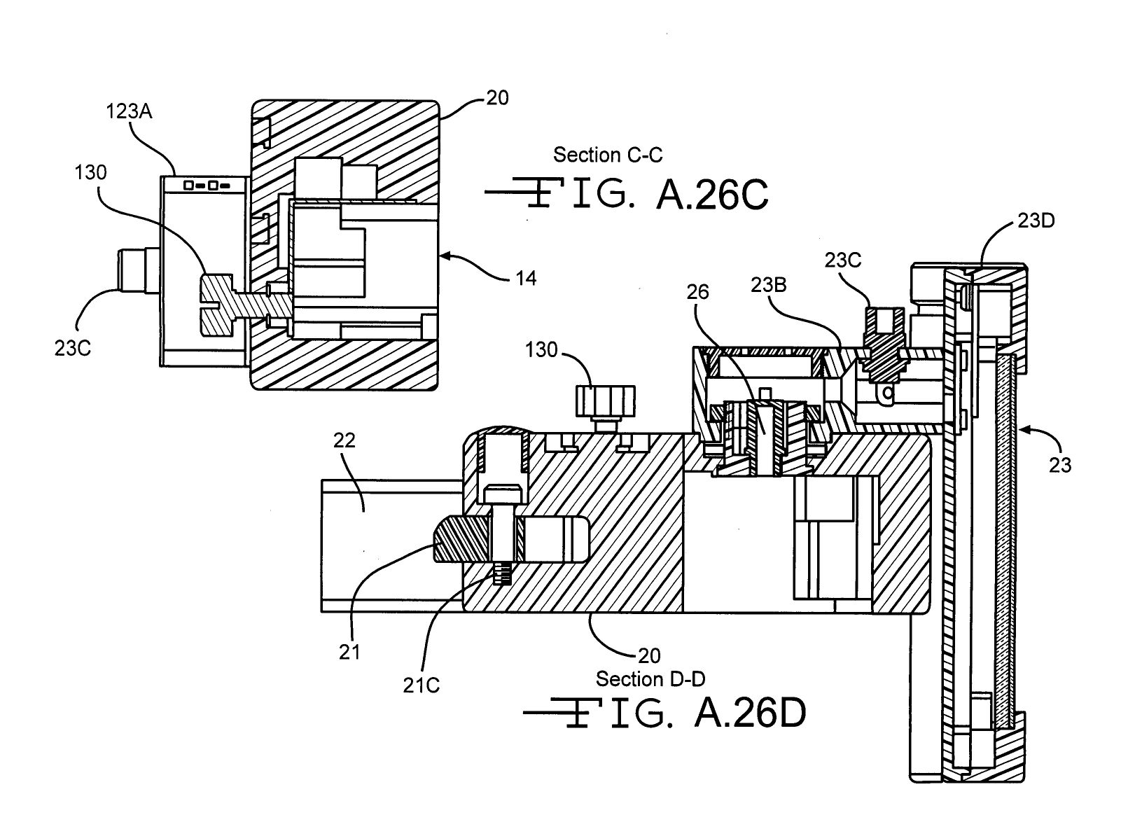

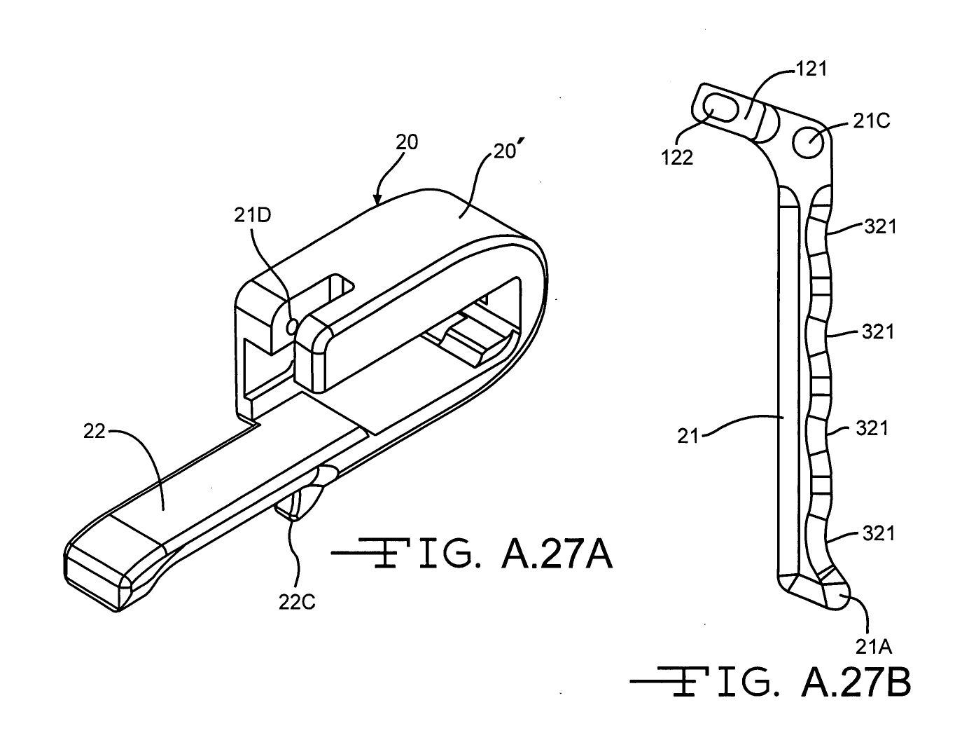

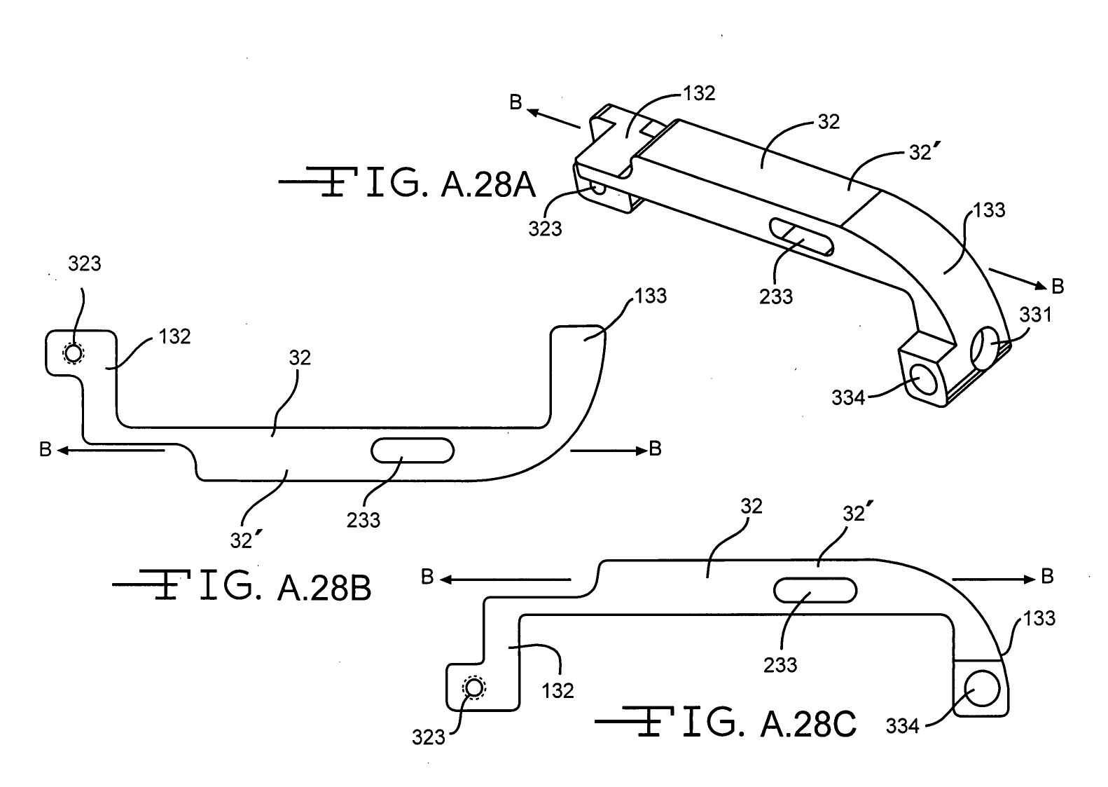

The electronic connection between the display means 323A and the camera means 28, 401 can suitably include any combination of wired and wireless electronic connections. As illustrated in FIG. 1, the cable 404 provides a wired connection between the laryngoscope blade 400 second camera means 401 and the display means 323A, and an internal cable (not shown) provides a wired connection between the first camera means 28 and the display means 323A through the stylet 12 and the handle 20. Similarly as shown in FIG. 9, an external cable 503 can be used to provide a wired connection between the display means and the first camera means 28 (again via an internal cable through the stylet 12 and the handle 20), for example in an embodiment where a display means 501 is not mounted on the intubation device 10. If the wireless laryngoscope blade 420 of FIG. 4 were used in the system shown in FIG. 1 or 9, then the cable 404 can be omitted and a wireless electronic connection can be established between the second camera means 401 and the display means 323A/501 (i.e., when the display means also includes a complementary wireless transmitter/receiver). If a wireless electronic connection is provided between two system components, a wired electronic connection also can be provided between the components to serve as a redundant/alternate connection means.

In addition to the electronic connection between the display means and the camera means of the intubation system, the components of the system can be physically connected (e.g., mounted to each other) or separated (e.g., physically disconnected other than possibly a wire/cable providing the electronic connection between the components) as desired. For example, as shown in FIG. 1, the display means 323A is mounted on the handle 20 of the endotracheal intubation device 10 so that the first view screen 323A and the second view screen 323C are viewable together by the operator M during use. In an analogous embodiment (not shown), the display means 323A can be mounted adjacent the blade proximal end 400A (e.g., mounted on the proximal end 400A of the blade 400 or on a blade handle at the proximal end 400A of the blade 400) so that the first view screen 323B and the second view screen 323B are viewable together by the operator M during use. As shown in FIG. 9, a display means 501 can be mounted on a structure other than an endotracheal intubation device 600 or a laryngoscope blade 400 so that a first view screen 501A and a second view screen 501B are viewable together by the operator during use. Such other structure can include a pole 500 such as a vertical pole that is mounted in a fixed position or that has a movable base (e.g., a wheeled base) for convenient positioning by the operator M for viewing. The display means 501 can be adjusted for height and angle by the operator M. The use of the pole 500 makes the viewing comfortable for the operator M. As shown, a cable 503 electronically connects the endoscope 600 with the display means 501; however, a wireless connection could be used instead of or in addition to the cable 503 (e.g., with a wireless transmitter/receiver mounted in the intubation device 600, such as in the handle or proximal region of the stylet).

FIGS. 1, 6, and 9 illustrate a suitable method for intubating a patient P using the disclosed intubation system. The endotracheal intubation system in any of its various embodiments (e.g., with the intubation device 10, the blade 400, and the display means 323A) is provided and an endotracheal tube 25 is mounted to the stylet 12 of the intubation device 10. The operator M controls the laryngoscope blade 400 with one hand to insert the blade 400 into the patient's airway passage and to manipulate the blade 400 to lift the patient's epiglottis. The operator M views the second image of the patient's airway passage on the second view screen 323C while inserting and manipulating of the laryngoscope blade 400 to ensure that the blade is correctly and safely inserted and positioned in the patient's airway passage. Once inserted, the laryngoscope blade 400 defines an open passageway between a surface of the laryngoscope blade 400 and surrounding soft tissue in the patient's airway passage. Similarly, the operator M controls the intubation device 10 with the other hand to place the endotracheal tube 25 in the patient's airway passage while viewing the first image of the patient's airway passage on the first view screen 323B during placement of the endotracheal tube 25. Placement of the endotracheal tube 25 suitably includes inserting/advancing the endotracheal tube 25 through the open passageway defined by the blade 400 and patient's internal soft tissue and further into the to the patient's trachea E. Once in place, either or both of the blade 400 and the intubation device 10/stylet 12 can be removed from the patient P.

The combination of the first camera means 28 for the stylet 12 and the second camera means 401 for the blade 400 provides several advantages that allow the operator M to perform an intubation process rapidly and safely on the patient P. Generally, the blade 400 is inserted into the patient's airway (e.g., to its final, desired location in the pharynx where it holds/lifts the epiglottis but is upstream of/above the vocal cords, larynx, and trachea) prior to insertion of the stylet 12/endotracheal tube 25 into the patient's airway passage. However, the blade 400 and the stylet 12/tube 25 can be advanced together or separately in any desired combination. Although the blade 400 defines an open passageway between its curved surface and the surrounding soft tissue in the patient's airway passage, subsequent insertion of the stylet 12/tube 25 through the open passageway can nonetheless injure the patient's soft tissue (e.g., roof of the mouth/palate and/or the oral/laryngeal pharynx wall) if care is not taken to avoid or minimize damaging contact between the two. In this case, the first camera means 25 and corresponding first image permits the operator to safely navigate the open passageway defined by the blade 400 as well as to properly locate/insert the tube 25 into the trachea E without damaging any soft tissue prior to reaching the trachea E.

Although the first camera means 28 permits safe and reliable insertion of the stylet 12/tube 25 into the trachea E, it does not necessarily provide reliable information about the degree of insertion of the tube 25 into the trachea E. For example, the degree of insertion of the tube 25 can be expressed as a linear insertion distance of the distal end of the tube 25 into the trachea E relative to a selected internal anatomical reference point in the particular patient (e.g., the location of the vocal fold/vocal cords). The total linear distance of the curved path of a particular patient's airway (e.g., extending through the mouth, pharynx (oral, laryngeal parts), larynx, and trachea) varies not only with the anatomy of the particular patient but also with the relative position of the patient's head, neck, and torso. The endotracheal tube 25 generally has visible length indicia (e.g., more generally any length-indicating means such as externally printed or otherwise externally viewable indicia or markings) that indicate to the operator M the position of any point along the length of the tube 25 relative to the distal and/or proximal ends of the tube. However, normal patient-to-patient variation in the length of the patient's airway means that viewing a tube 25 length index visible to the operator M (e.g., on a portion of the tube 25 either inside the mouth or immediately outside the mouth) does not reliably indicate the degree of insertion of the tube 25 into the trachea E. Another complicating factor is the fact that the length of the airway extends/contracts as the head is moved relative to the torso. For example, in a normal adult, the airway is about 2 cm longer when the head is tilted forward/down (e.g., when the chin is touching or near its closest point to the chest) as compared to the length when the head is upright and/or tilted backward. Thus, depending on the particular position of the patient's head during the intubation process (e.g., head-down, head-back, or head-up such as in the horizontal supine position), the position of an inserted tube 25 may slide or otherwise change if the head is subsequently moved. For example, a change in the position of the patient's neck can create pushing or pulling forces to move the distal end of the inserted tube 25 either further away from or closer to the vocal cords, respectively (e.g., due to frictional or pinching/compression forces between the tube 25 and a portion of the patient's airway above the vocal cords). To address such cases, it is desirable to control the degree of insertion of the tube 25 into the trachea E so that inadvertent post-intubation slippage or other displacement of the tube 25 does not cause the tube 25 to become dislodged from the trachea E (i.e., at least a distal portion of the tube 25 remains in the trachea E even if the tube 25 does move from the location selected by the operator M during the intubation process).

As seen in FIG. 6, the final ultimate insertion location of the blade 400 is such that at least a portion of the patient's vocal cords, larynx, and/or trachea E are in the field of view of the second camera means 401. Further, as the distal end 112 of the stylet 12 is advanced past the distal end 400B of the blade 400, the endotracheal tube 25 (e.g., the portion at or adjacent the distal end thereof) also enters the field of view of the second camera means 401 (i.e., the area generally in front of or extending beyond the distal end 400B of the blade 400). In this way, the operator M can use the second camera means 401 to view the length indicia on the tube 25 and control the degree of insertion of the tube. Specifically, once the tube 25 passes the vocal cords and enters the larynx and trachea, the operator M can simultaneously view (i) the first image from the first camera means 28 to view patient soft tissue immediately in front of the stylet 12 to ensure continued safely advancement of the tube 25 into the larynx and trachea and (ii) the second image from the second camera means 401 to view the tube 25 length index that is above the vocal cords. This provides the operator M with an accurate measurement of the degree of tube insertion expressed as a linear distance past the vocal cords into the trachea E. Suitably, the degree of insertion past the vocal cords is about 3 cm to about 5 cm, but more generally can be at least about 1 cm, 2 cm, 3 cm, 4 cm, 5 cm, or 6 cm and/or up to about 2 cm, 3 cm, 4 cm, 5 cm, 6 cm, 8 cm, or 10 cm, Further, once the tube 25 is in its desired location and has the selected degree of insertion, the second camera means 401 can be used as the blade 400 is withdrawn from the patient to view the tube 25 length indicia and measure the relative location of other internal airway structures (e.g., epiglottis, tongue).

As further shown in FIGS. 1 and 9, a syringe S is used to inflate a cuff on the endotracheal tube 25 to prevent reflux into the airway, as is well known to those skilled in the art. As can be seen from the preceding disclosure, the screen could be on the blade handle and the tube stylet connected to this screen by a cord or a wireless connection.

### Endotracheal Intubation Device

FIGS. A.1-A.28 illustrate a suitable endotracheal intubation device for use with the endotracheal intubation system described above (e.g., as the endoscope 10 shown in FIG. 1).

Referring to FIGS. A.1-A.7, an exemplary endotracheal intubation device is shown having a hand grip 20 with a trigger 21 for convenient articulation of an articulation section 13. FIG. A.1 illustrates endotracheal intubation device 10 in use on a patient P. Device 10 is being operated by a medical professional M to access patient P's trachea E. In an exemplary embodiment, device 10 includes a detachable stylet assembly 11 which comprises an elongated tube 12. Stylet assembly 11 is adapted to connect with a hand grip 20. In FIG. A.2, elongated tube 12 defines: a longitudinal axis A-A, a proximal end 12A for detachably mounting the stylet to hand grip 20, and a distal end 12B for entering the trachea E of patient P. Tube 12 comprises an articulation section 13 adjacent distal end 12B. Section 13 is adapted to curve into trachea E upon actuation of trigger 21 from hand grip 20. As shown in FIG. A.1, articulation section 13 is curved into trachea E of the patient since it is being actuated by the trigger 21 on hand grip 20. The hand grip 20 comprises a trigger 21 that is squeezed by professional M to actuate section 13. Mounted on tube 12 is a tube stop 24 comprising an adjustment knob 24A and adapted to allow for mounting of an endotracheal tube 25 over and around the elongated tube 12. In a particular embodiment, device 10 is used to insert and place endotracheal tube 25 into the patient P to clear the trachea E and then device 10 is subsequently removed leaving endotracheal tube 25 in place for further procedures to be performed.

Detachable stylet assembly 11, as shown detached from hand grip 20 and alone in FIGS. A.3A and A.3B, comprises an actuator housing 14. Housing 14 defines a substantially rectangular cross section and encloses an actuating assembly 15, as shown in the various magnified and exploded views of FIGS. A.9-A.17 discussed in greater detail below. Actuator housing 14 is mounted on and adjacent to proximal end 12A of tube 12. Actuating assembly 15 comprises a connection means 16 for engaging hand grip 20. Connection means 16 extends out from the enclosure of housing 14 on an opposite side from where tube 12 extends out of housing 14. Connection means 16 can be a stem that is operable to engage trigger mechanism 21 of hand grip 20. This engagement occurs when stylet assembly 11 is connected with hand grip 20. Actuator housing 14 includes a back plate 17 for protecting internal components of actuator assembly 15 from the external environment. Back plate 17 includes an extension 17A that lays flush with hand grip 20 when stylet 11 is attached. This provides external protection for connection means 16.

Medical professional M typically stands at the head of patient P when intubating. As tube 12 is inserted into the trachea E of patient P, medical professional P can squeeze trigger 21 which is connected through connection means 16 to actuator assembly 15. Actuator assembly 15 connects to a control wire 18 (shown in FIGS. A.9, A.10, A.12, A.13, and A.17). Control wire 18 is mounted within actuator housing 14 and connected to the connection means 16. Control wire 18 extends through tube 12 to distal end 12B and is adapted to curve the articulation section 13 upon actuation. Actuation occurs when trigger 21 is squeezed. In an exemplary embodiment, articulation section 13 is curveable via a vertebrae configuration or a Nitinol tube as described with respect to U.S. patent application Ser. No. 12/148,050 filed Apr. 16, 2008. While the elongated tube 12 can be constructed of stainless steel, polymer or other sturdy material, in some preferred embodiments it is constructed of a shape memory alloy (SMA). Any shape memory alloy such as a copper-zinc-aluminum, copper-aluminum-nickel, and nickel-titanium (NiTi) alloys can be used, such as, but not limited to Nitinol. The articulation or curveable portion 13 of elongated tube 12 can be constructed of a shape memory alloy such as Nitinol. The shape memory alloy (SMA) of the articulation section 13 will flex when the trigger 21 is squeezed, and then will return to its original conformation when the trigger 21 is released due to the tendency of the SMA to spring back to a less curved conformation.

In an exemplary embodiment, elongated tube 12 comprises at least one LED light 19 mounted adjacent the distal end 12B of elongated tube 12 as shown in FIG. A.4E. LED light 19 is adapted to illuminate a pathway for stylet 10 to enter the trachea E. FIG. A.2 illustrates a side view of device 10 with trigger 21 at rest and thus articulation section 13 in a substantially straight configuration. LED light 19 is typically mounted in the tip 112.

In an exemplary embodiment as shown in FIGS. A.1, A.2, and A.4A-A.4D hand grip 20 comprises a display means 23. Display means 23 can be pivotably mounted on hand grip 20 to allow for rotation to a desired viewing position. Display means 23 can be any viewing monitor such as an LCD screen. As described above, the display means 23 can include a single monitor screen 123 (e.g., as shown in FIG. A.23D) that provides simultaneous split-screen display and viewing (e.g., in a side-by-side or other arrangement) of the first and second images on first and second virtual monitor screens of the single monitor screen 123. As shown in FIG. A.1, display means 23 pivots towards medical professional M for better viewing. FIG. A.4C and A.4D are side views of device 10 with monitor 23 in opposite configurations illustrating that the monitor can rotate or pivot approximately 180 degrees. FIG. A.4B shows the trigger 21 and articulation section 13 in an actuated state of trigger 21′ and articulation 13′, i.e., curved when trigger 21 is squeezed towards stationary handle 22 of handgrip 20 and represented in dotted lines. As shown in FIG. A.2 and FIG. A.6, in an exemplary embodiment, display means 23 is connected to a base extension 23A which connects to hand grip 20 on pivot point 23B. Pivot point 23B can be any means for providing a desired pivot such as a screw or bolt. In a particular embodiment base extension 23A is constructed to define a substantially triangular geometry thus providing adequate support for the monitor and convenient pivoting along pivot point 23B.

In an exemplary embodiment, detachable stylet 11 comprises an electrical connection 26 illustrated in FIG. A.3A. Electrical connection 26 provides a port and connection to a camera having a lens 28 (schematically shown in FIG. A.4E) mounted in the tip 112. In a particular embodiment, the camera is a CMOS chip having optics. Electrical connection 26 is adapted to receive a mating connector mounted inside of hand grip 20. Connecting stylet 11 to handgrip 20 allows for connection of camera 28 to display means 23 via electrical connection 26. The camera is connected to electrical connection 26 through one or more wires that run through the interior length of elongated tube 12. When connected or coupled to each other, display means 23 can display a pathway through the trachea via the camera. This provides for convenient steering and guidance for the medical professional to direct the tube into a desired location.

Typically, stylet 11 is disinfected in a disinfecting fluid prior to use. Submerging stylet 11 in a liquid for any period of time can damage any of the electrical components, including electrical connection port 26 and the camera. Thus, in an exemplary embodiment, device 11 comprises a soak cap 27. Soak cap 27 is adapted to protectively mount over and around electrical connection port 26. Exposure to liquid is substantially prevented when soak cap 27 is mounted over connection port 26 thus allowing for convenient disinfection of stylet 11. If stylet 11 is submerged in a disinfection liquid, the electrical components are protected by soak cap 27. Typically, soak cap 27 is mounted on one side of hand grip 20 as shown in FIG. A.2. Soak cap 27 rests on the outer surface of hand grip 20 until it is needed to cover the electrical connection 26 of stylet 11 as shown in FIG. A.3A. Typically electrical connection 26 faces perpendicular to axis A-A on interior face 17B of housing 14. In a particular embodiment, soak cap 27 lies flush with face 17B when mounted over electrical connection 26.

FIGS. A.5-A.7 illustrate magnified opposite side views and a trigger side view of hand grip 20 having display means 23 and mounted to stylet 11. Actuator housing 14 forms an enclosure around the actuator assembly and proximal end of elongated tube 12. In a particular embodiment, back plate 17 is mounted onto housing 14 by several mounting features 29 such as screws or bolts. Hand grip 20 comprises: (i) a grip housing 20′, (ii) a trigger 21 as a pivotable lever extending from housing 20′ towards distal end 12B of elongated tube 12 and away and nonparallel with respect to longitudinal axis A-A; and (iii) a stationary handle 22 as base for squeezing trigger 21 towards handle 22 when engaged. Base handle 22 extends towards distal end 12B and parallel with respect to axis A-A. Trigger 21 is mounted onto housing 20′ on a pivot feature 21C.

In an exemplary embodiment, trigger 21 and handle 22 each define a curving lip 21A and 22A at distal ends 21B and 22B, respectively. Lips 21A and 22A serve as terminating ends of trigger 21 and handle 22 such that a user can conveniently feel where to properly place his hand when using device 10. Although shown as terminating ends, lips 21A and 22A can be located anywhere along their respective structure since their intention is to provide indication to a particular hand position. Lips 21A and 22A also serve as structural stops to substantially deter slipping of the hand. Typically lips 21A and 22A face away from each other.

FIG. A.6 illustrates an opposite side view of FIG. A.5 of hand grip 20 with display means 23 mounted thereon. In a particular embodiment, display means 23 is mounted on a pivotable extension 23A on a pivot point 23B. In a particular embodiment, a video-out port 23C is mounted on extension 23A. Typically, port 23C is an RCA composite port adapted to allow for coupling or connection to an external display means or monitor such as a computer or LCD screen. Display means 23 can further comprise a power source 23D such as a battery. Housing 20′ can be securely held together by a securing means 20″ such as a screw or bolt.

FIGS. A.8-A.17 illustrate various exploded views of the internal components of both hand grip 20 and actuator assembly 15. FIG. A.8 illustrates a side view from the stylet 11 side of device 10 with the grip housing 20′ removed. FIG. A.9 illustrates device 10 with the grip housing 20′ and actuator housing 14 removed from the opposite side of FIG. A.8. Trigger 21 is connected to a lever member 121 at pivot feature 21C. Lever member 121 extends substantially perpendicular to the axis A-A and defines an opening 122. In an exemplary embodiment, opening 122 defines an oblong geometry to all for sliding of the lever member during squeezing, i.e. actuation, of the trigger 21.

Trigger 21 engages connector linkage 32 through opening 122 of lever member 121 by a pin 124. Connector linkage 32 includes a distal end 132 and a proximal end 133. The distal end connects to trigger 21 through a connection means, such as a pin, screw, or bolt, in opening 122. As shown in FIG. A.10, linkage 32 includes an elongated back bone 32′ that extends along a longitudinal axis B-B which is parallel with axis A-A. Extending perpendicular to axis B-B are distal end 132 and proximal end 133. Back bone 32′ defines a pair of guidance openings 233. Openings 233 are substantially elongated ovals that receive guide pins 33. Guide pins 33 are mounted on the inner surface of grip housing 20′ and extend through the openings 233. Pins 33 provide structural guidance to linkage 32 during actuation. Typically, when trigger 21 is squeezed towards base handle 22, lever member 121 pivots to apply force to linkage 32 at the connection at distal end 132. Linkage 32 is connected to lever member 121 through a pin 124 in opening 122. Linkage 32 translates along the A-A axis away from elongated tube 12. Guide pins 33 provide structural support to linkage 32 and thus backbone 32′ translates along axis B-B. A coil spring 31 is positioned between proximal end 133 of linkage 32 and support stop 30. The spring is operable to return linkage 32 and thus trigger 21 back to resting position when the force on the trigger is removed.

FIGS. A.9-A.14 illustrate the internal components of actuator assembly 15. Connection means 16 is part of actuator assembly 15. In a particular embodiment, actuator assembly 15 is constructed within actuator housing 14 of detachable stylet 11. In a further embodiment, device 10 is constructed as an entire assembly without the detachable feature. Connection means 16 can be any means to secure connection of actuator assembly 15 to linkage 32 such as a stem, protruding pin or bullet shaped extension. Connection means 16 engages a hole or opening defined in proximal end 133 of linkage 32. Connection means is attached to a plunger 34 that slides within a chamber 35. Chamber 35 abuts against a seal 36 to prevent liquid contamination during actuation. Plunger 34 is connected to control wire 18. During actuation, i.e., trigger 21 is being squeezed, lever 121 acts upon linkage 32 which translates away from trigger 21. As linkage 32 compresses spring 31, it acts upon connector means 16 which pulls plunger 34 to translate through chamber 35. Plunger 34 pulls control wire 18 which then causes tube 12 to curve at the articulation section 13. This mechanism provides a user with controlled curving and movement through the squeezing of trigger 21.

FIGS. A.11-A.14 illustrate device 10 having a retaining plate 37. Retaining plate 37 is mounted within hand grip 20 and provides structural support for electrical connection 26 and pivot point 23B. Mating electrical connection 26′ is shown in FIG. A.11 within retaining plate 37 and is adapted to engage electrical connection 26 mounted within actuator housing 14. Seal 36 abuts against seal support 38 which operates as a stop for forming the seal during actuation. FIGS. A.15-A.17 illustrate top views of device 10 with various components removed to illustrate component configuration.

FIGS. A.18-A.19 illustrate a front view of the display means 23. Display means 23 can be any monitor having a screen 123. In a particular embodiment, monitor 123 is a LCD screen. Display means 23 includes a power source 23D such as a battery. In a particular embodiment, as shown in FIGS. A.20-A.23, a device 210 is provided having hand grip 220, trigger 221, and base handle 222. Hand grip 220 is adapted to attach to a detachable stylet 11 and function in the same manner as previously described with respect to hand grip 20 above. Hand grip 220 defines a monitor rotation slot 225 adapted to allow for rotating of display means 223. Display means 223 is mounted on a swivel point 226 of a rotating arm 224. Swivel point 226 allows for a user to position display means 223 in a variety of desired orientations. The rotation through slot 225 in combination with the swivel point 226 provides for convenient repositioning of display means 223 for optimal viewing angles. FIG. A.21 and A.22 illustrate exemplary positions of display means 223 with respect to hand grip 220.

In an exemplary embodiment, the video signal from the display means is broadcast wirelessly. The handle functions as a grounding plate for the dipole antenna of the wireless transmitter. The user can function as a grounding plate and thus facilitating clarity of the wireless signal to the receiver.

FIGS. A.23A-A.28C illustrate further particular exemplary embodiments of endotracheal intubation devices with a stylet, hand grip, and display monitor according to the present disclosure. Like numerals are used to describe like features with respect to the embodiments described in FIGS. A.1-A.22. Any differences from those embodiments are described below.

FIGS. A.23A-A.23D illustrate an exemplary endotracheal intubation device 10 having a hand grip 20 with a trigger 21 for convenient articulation of an articulation section 13. FIG. A.23A illustrates a first side view of endotracheal intubation device 10 showing a pivot mount 23A for monitor 23. Monitor 23 is connected to grip housing 20 via pivot mount 23A. Pivot mount 23A allows for manual pivoting of monitor 23 about pivot point 23B. The pivot mount 23A is mounted on one side of grip housing 20.

Device 10 can be operated by a medical professional to access patient trachea or allow for viewing of the vocal chords and trachea. FIG. A.23B illustrates a trigger side or bottom side view of device 10. FIG. A.23C illustrates a stylet side view showing the engagement of stylet assembly 11 with grip housing 20. FIG. A.23D illustrates a front view showing monitor 23 and trigger 21 extending away and upwardly at an angle with respect to monitor 23. Monitor 23 comprises a display means 123 and a power source 23D. Display means 123 can be any visual display technology such as a liquid crystal display (LCD). Typically, power source 23D is comprised of lead acid or lithium ion batteries. In a particular embodiment, monitor 23 comprises a low battery light indicator 324, typically an LED. Monitor 23 is in electrical connection with stylet 11 through electrical connection 26 (FIG. A.24B). Electrical connection 26 is coupled through electrical wires to a camera mounted in a tip portion 112 of stylet 11. The camera comprises an external lens 28 shown in the distal tip view of FIG. A.24G. Typically lens 28 is mounted adjacent at least one, but often two LEDs 19 to provide light in use. The camera allows for the medical professional to view the pathway into the patient via the monitor 123. This allows for more accurate tube 12 placement of the stylet assembly 11 and for avoiding damage to sensitive and crucial anatomy such as the vocal chords.

FIGS. A.25A-A.25C illustrate hand grip 20 detached from stylet assembly 11. Grip 20 comprises a trigger 21 which pivots about a pivot pin 21C (FIG. A.25C). Grip 20 defines a pair of receiving holes 21D (FIG. A.27A) for receiving pin 21C. This allows for trigger 21 to pivot about pin 21C when squeezed. A stand alone trigger 21 is shown in FIG. A.27B. Trigger 21 includes finger grooves 321 for easy and effective gripping. Hand grip 20 includes a stationary handle 22 as a base. Base handle 22 extends away from housing 20 to provide a gripping means for a user. Typically, a medical professional can place his hand against base 22 while wrapping his fingers around the pivotable trigger 21. Trigger 21 is connected to housing 20 such that it, retracts away from base 22 while at rest.

Trigger 21 is connected to hand grip 20 through pin 21C. Trigger 21 comprises a lever member 121. Lever 121 extends at an angle from pivot pin 21C inside hand grip 20. A cam 122 is defined on lever 121 (See FIG. A.27B) which allows for connection to linkage 32 (FIG. A.28A) through a translating pin 124. Cam 122 receives pin 124 which connects to linkage 32 at pin hole 323 (FIGS. A.28A-A.28C). When trigger 21 is squeezed, pin 124 moves within cam 122 and translates linkage 32 linearly towards monitor 23 along axis B-B (FIG. A.28B). Linkage 32 can also be referred to as a bolt. A spring 31 (shown in cross section view A-A of housing 20 in FIG. A.26A) abuts against linkage 32 in a spring cavity 331. Typically spring 31 is a coil spring and returns trigger 21 to its original rest position once a squeezing force is removed. Spring 31 abuts against a stop 30 mounted within grip 20 (See FIG. A.26A).

Base handle 22 comprises an upper stop 22C. Stop 22C extends perpendicular to an axis defined by handle 22. Typically, stop 22C also curves slightly thereby substantially resembling a shark fin geometry as shown in the side view of FIG. A.25C. The stop 22C is detectable to the touch and slightly forms around the hand of the user's grip. The fin configuration substantially prevents unintended slippage. Moreover, the stop 22C can serve as an anchor or abutment for the user to secure his grip while pulling on trigger 21.

In an exemplary embodiment, device 10 comprises a detachable stylet assembly 11 as shown detached in FIGS. A.24A-A.24C. Stylet 11 is comprised of an elongated tube 12 extending from actuator housing 14. Stylet 11 can also be referred to as a “working length” and is adapted to connect into hand grip 20. Typically, stylet 11 snaps into place in hand grip 20 but can also be secured by a screw 130 as shown in FIG. A.25C. When mounted in grip 20, electrical connection 26 is coupled to a receiving section in grip 20 and forms an electrical connection between the camera and monitor 23.

FIG. A.24A illustrates a perspective view of stylet 11. Stylet assembly 11 is adapted to connect with a hand grip 20 as shown in FIG. A.23C. Elongated tube 12 defines a proximal end 12A for detachably mounting the stylet to hand grip 20, and a distal end 12B for entering the trachea of a patient. Tube 12 comprises an articulation section 13 adjacent distal end 12B. Section 13 is adapted to curve into a trachea upon actuation of trigger 21 from hand grip 20. The hand grip 20 comprises a trigger 21 that is squeezed by a professional to actuate section 13. Mounted on tube 12 is a tube stop 24 (FIGS. A.24A-A.24C, 24F) comprising an adjustment knob 24A and adapted to allow for adjustable mounting of an endotracheal tube 25 over and around the elongated tube 12. In a particular embodiment, device 10 is used to insert and place an endotracheal tube into the patient to clear the trachea and then device 10 is subsequently removed leaving the endotracheal tube (not shown) in place for further procedures to be performed.

Mounted on the same side of grip housing 20 is soak cap 27. Soak cap 27 is threadedly mounted on grip housing 20 so it can be removed conveniently when needed. Soak cap 27 covers electrical connection 26 (described below with respect to FIG. A.24B) to prevent liquid contamination and to keep electrical connection 26 dry during disinfection. Often components of device 10 are submerged in a cleaning solution to disinfect components. Certain electrical components such as electrical connection 26 must be covered and isolated from the liquid to prevent damage. Soak cap 27 can be screwed over electrical connection 26 and is configured to prevent liquid intrusion and contact to connection 26. During operation of device 10, soak cap 27 is mounted on the side of housing 20 as shown in FIG. A.23A for use with the disinfection of the stylet assembly 11 (FIGS. A.24A-A.24C). Mounting soak cap 27 on grip housing 20 significantly prevents loss or misplacement of soak cap 27 when soak cap 27 is not in use. In an exemplary embodiment, soak cap 27 is made a unique and distinguishing color, such as orange to stand out from the other components. FIG. A.24E illustrates cross section B-B of FIG. A.24C with soak cap 27 mounted on and covering connection 26.

Detachable stylet assembly 11, as shown detached from hand grip 20 and alone in FIGS. A.24A-A.24C and cross section views FIGS. A.24D-A.24G, comprises an actuator housing 14. Housing 14 defines a substantially rectangular cross section and encloses an actuating assembly 15. Actuator housing 14 is mounted on and adjacent to proximal end 12A of tube 12. Actuating assembly 15 comprises a connection means 16 (FIGS. A.24B and A.24F) for engaging hand grip 20. Connection means 16 extends out from the enclosure of housing 14 on an opposite side from where tube 12 extends out of housing 14. Connection means 16 can be a stem that is operable to engage trigger mechanism 21 of hand grip 20. Stem 16 resembles a bullet shape and allows for mechanical engagement between the grip 20 (through linkage 32) and the articulation section 13. This engagement occurs when stylet assembly 11 is connected with hand grip 20. Actuator housing 14 includes a back plate 17 for protecting internal components of actuator assembly 15 from the external environment. Back plate 17 includes an extension 17A that lays flush with hand grip 20 when stylet 11 is attached. This provides external protection for connection means 16.

A medical professional M typically stands at the head of a patient P when intubating (shown in FIG. A.1). As tube 12, with an endotracheal tube, is inserted into the trachea E of the patient P, the medical professional M can squeeze trigger 21 which is connected to actuator assembly 15 through linkage 32 and connection means 16. Actuator assembly 15 connects to a control wire 18 as shown in FIG. A.24B. Control wire 18 is mounted within actuator housing 14 and connected to the connection means 16. Control wire 18 extends through tube 12 to distal end 12B and is adapted to curve the articulation section 13 upon actuation. Actuation occurs when trigger 21 is squeezed. In an exemplary embodiment, articulation section 13 is curveable via a vertebrae 213 configuration as shown in FIG. A.24D or a Nitinol tube as described with respect to U.S. patent application Ser. No. 12/148,050 filed Apr. 16, 2008 incorporated hereby in its entirety. Cross section A-A of section 13 is shown in the exemplary embodiment of FIG. A.24D illustrating the vertebrae 213 embodiment. Each vertebrae section 213 is adapted to allow for section 13 to curve upon actuation.

While the elongated tube 12 is preferably constructed of a rigid stainless steel tube, a polymer or other sturdy material, in some preferred embodiments can be used. The tube can be flexible or rigid. The actuating section 13 is preferably constructed of a shape memory alloy (SMA). Any shape memory alloy such as a copper-zinc-aluminum, copper-aluminum-nickel, and nickel-titanium (NiTi) alloys can be used, such as, but not limited to Nitinol. The articulation or curveable portion 13 of elongated tube 12 can be constructed of a shape memory alloy such as Nitinol with cuts to allow for articulation. The shape memory alloy (SMA) of the articulation section 13 flexes when the trigger 21 is squeezed, and then will return to its original conformation when the trigger 21 is released due to the tendency of the SMA to spring back to a less curved conformation. FIGS. A.23A-A.23C illustrate device 10 with trigger 21 at rest and thus articulation section 13 in a substantially straight configuration.

In an exemplary embodiment, elongated tube 12 comprises at least one LED light 19 mounted adjacent the distal end 12B of elongated tube 12 as shown in FIG. A.24G. LED light 19 is adapted to illuminate a pathway for stylet 10 to enter the trachea. LED light 19 is typically mounted in the tip 112 as shown in the distal tip view of FIG. A.24G.

Monitor 23 can be pivotably mounted on hand grip 20 to allow for rotation to a desired viewing position. Monitor 23 can pivot towards a medical professional for better viewing. In an exemplary embodiment, the monitor can rotate or pivot approximately 180 degrees. As shown in FIG. A.23A-A.23C and 12-14 monitor 23 is connected to a base extension 23A which connects to hand grip 20 on pivot point 23B. Pivot point 23B can be any means for providing a desired pivot such as a screw or bolt. In a particular embodiment base extension 23A is constructed to define a substantially triangular geometry thus providing adequate support for the monitor and convenient pivoting along pivot point 23B.

Detachable stylet 11 comprises an electrical connection 26 illustrated in FIG. A.24B. Electrical connection 26 extends outward from housing 14 substantially perpendicular to a longitudinal axis defined by tube 12. Connection 26 engages an electrical receiving portion of grip housing 20 to connect with monitor 23. This allows for connection to a camera having a lens 28 (shown in FIG. A.24G) mounted in the tip 112. In a particular embodiment, the camera is a CMOS chip having optics. Connecting stylet 11 to handgrip 20 allows for connection of the camera to monitor 23 via electrical connection 26. The camera is connected to electrical connection 26 through one or more wires that run through the interior length of elongated tube 12. When connected or coupled to each other, monitor 23 can display a pathway through the trachea via the camera. This provides for convenient steering and guidance for the medical professional to direct the tube 12 into a desired location.

Actuator housing 14 forms an enclosure around the actuator assembly 15 and proximal end of elongated tube 12. In a particular embodiment, back plate 17 is mounted onto housing 14 by several mounting connectors 29 such as screws or bolts.

FIGS. A.25A-25C and A.27A illustrate an exemplary hand grip 20. Hand grip 20 comprises: (i) a grip housing 20′, (ii) the trigger 21 as a pivotable lever extending from housing 20′ towards distal end 12B of elongated tube 12 and away and nonparallel with respect to longitudinal axis A-A as shown in FIG. A.23C; and (iii) a stationary handle 22 as base for squeezing trigger 21 towards handle 22 when engaged. Base handle 22 extends towards distal end 12B and parallel with respect to axis A-A. Trigger 21 is mounted onto housing 20′ on a pivot pin 21C.

In an exemplary embodiment, trigger 21 and handle 22 each define a curving lip 21A and 22A at distal ends 21B and 22B, respectively. Lips 21A and 22A serve as terminating ends of trigger 21 and handle 22 such that a user can conveniently feel where to properly place his hand when using device 10. These lips 21A and 22A are also referred to as “stops” and typically lip 22A is substantially curved to prevent unintended hand slipping while device 10 is in use. Although shown as terminating ends, lips 21A and 22A can be located anywhere along trigger 21 and handle 22 since their intention is to provide indication to a particular hand position. Lips 21A and 22A also serve as structural stops to substantially deter slipping of the hand. Typically lips 21A and 22A face away from each other and are at substantially right angles with respect to trigger 21 and handle 22 respectively.

FIGS. A.23A and A.25C illustrate a side view of hand grip 20 with monitor 23 mounted thereon. In a particular embodiment, monitor 23 is mounted on a pivot mount 23A on a pivot point 23B. In a particular embodiment, a video-out port 23C is mounted on mount 23A. Typically, port 23C is an RCA composite port adapted to allow for coupling or connection to an external display means or monitor such as a computer or LCD screen. Monitor 23 further comprises a power source 23D such as a battery. Housing 20′ can be securely held together by a securing means 130 such as a screw or bolt.

FIGS. A.24D-A.24G and A.26A-A.26D illustrate various exploded views of the internal components of both hand grip 20 and actuator assembly 15. FIGS. A.26A and A.27B show trigger 21 and extending lever 121 defining a cam 122 for receiving a translating pin 124. FIG. A.26A is a cross section A-A of FIG. A.25B. Lever 121 extends substantially perpendicular to the axis of handle 22 and defines cam 122. In an exemplary embodiment, cam 122 defines an oblong geometry to all for sliding of the lever member during squeezing, i.e. actuation, of the trigger 21. Pin 124 also connects trigger 21 to linkage 32.

Trigger 21 engages connector linkage 32 (shown alone in FIG. A.28A, A.28B, and A.28C) through cam 122 of lever 121 by pin 124 shown in FIG. A.26A. Connector linkage 32, shown in FIGS. A.26A and includes a distal end 132 and a proximal end 133. The distal end connects to trigger 21 through pin 124, in pin hole 323 of linkage 32. As shown in FIG. A.28A, linkage 32 includes an elongated back bone 32′ that extends along a longitudinal axis B-B which is parallel with the axis of handle 22. Extending perpendicular to axis B-B are distal end 132 and proximal end 133. Back bone 32′ defines an opening 233. Opening 233 defines a substantially elongated oval geometry that receives guide pin 33. As shown in FIG. A.26A, guide pin 33 is mounted on the inner surface of grip housing 20′ and extends through the opening 233. Pin 33 provides structural guidance to linkage 32 during actuation. Typically, when trigger 21 is squeezed towards base handle 22, lever 121 pivots to apply force to linkage 32 at the connection at distal end 132. Linkage 32 translates away from elongated tube 12. Guide pin 33 provides structural support to linkage 32 and thus backbone 32′ translates along axis B-B. A coil spring 31 is positioned between proximal end 133 of linkage 32 and support stop 30. The spring is operable to return linkage 32 and thus trigger 21 back to resting position when the force on the trigger is removed.

Connection means 16 is part of actuator assembly 15. In a particular embodiment, actuator assembly 15 is constructed within actuator housing 14 of detachable stylet 11. In a further embodiment, device 10 is constructed as an entire assembly without the detachable feature. Connection means 16 can be any means to secure connection of actuator assembly 15 to linkage 32 such as a stem, protruding pin or bullet shaped extension. Connection means 16 engages a hole 334 (FIG. A.28A) defined in proximal end 133 of linkage 32. Connection means 16 is attached to a plunger 34 that slides within a chamber 35 (FIG. A.24C and A.24F). Chamber 35 abuts against a seal 36 to prevent liquid contamination during actuation. Plunger 34 is connected to control wire 18. During actuation, i.e., trigger 21 is being squeezed, lever 121 acts upon linkage 32 which translates away from trigger 21. As linkage 32 compresses spring 31, it acts upon connector means 16 which pulls plunger 34 to translate through chamber 35. Plunger 34 pulls control wire 18 which then causes tube 12 to curve at the articulation section 13. This mechanism provides a user with controlled curving and movement through the squeezing of trigger 21 of articulation section 13.

In an exemplary embodiment, the video signal from the monitor is broadcast wirelessly. The handle functions as a grounding plate for the dipole antenna of the wireless transmitter. The user can function as a grounding plate and thus facilitating clarity of the wireless signal to the receiver.

While the present invention is described herein with reference to illustrated embodiments, it should be understood that the invention is not limited hereto. Disclosed numerical values and/or ranges may be interpreted either with or without the modifying term “about.” Those having ordinary skill in the art and access to the teachings herein will recognize additional modifications and embodiments within the scope thereof. Therefore, the present invention is limited only by the claims attached herein.

What is claimed is:

1. An endotracheal intubation system adapted for two-handed operation by a medical operator, the system comprising:
(a) an endotracheal intubation device comprising:
(i) a stylet adapted for mounting an endotracheal tube, the stylet having a proximal end and a distal end, and
(ii) a first camera means mounted to the stylet adjacent the distal end of the stylet and enabling viewing of a first image of a patient's airway passage on a first view screen;
(b) a laryngoscope blade for lifting a patient's epiglottis with simultaneous viewing of the patient's airway passage by the endotracheal intubation device, wherein
(i) the laryngoscope blade has a proximal end and a distal end, and
(ii) the laryngoscope blade comprises a second camera means mounted adjacent the distal end of the laryngoscope blade, the second camera means enabling viewing of a second image of the patient's airway passage on a second view screen; and
(c) a display means for simultaneously receiving and viewing visual images from at least two different camera means, wherein
(i) the display means is electronically connected to the first camera means and to the second camera means, and
(ii) the display means comprises the first view screen for viewing the first image and the second view screen for viewing the second image, the first view screen being positioned adjacent the second view screen on the display means;
wherein during use by a medical operator, a proximal end of the intubation device and the proximal end of the laryngoscope blade are each adapted to be manipulated together in two-handed operation by the operator's left and right hands together.

2. The endotracheal intubation system of claim 1, wherein the display means comprises one or more monitor screens adapted to independently and simultaneously display the first image and the second image.

3. The endotracheal intubation system of claim 2, wherein the one or more monitor screens are LCD screens.

4. The endotracheal intubation system of claim 1, wherein the display means comprises (i) a first monitor screen comprising the first view screen and being adapted to display the first image, and (ii) a second monitor screen comprising the second view screen and being adapted to display the second image.

5. The endotracheal intubation system of claim 1, wherein the display means comprises a first monitor screen, the first monitor screen comprising the first view screen and the second view screen as discrete regions of the first monitor screen and being adapted to independently and simultaneously display the first image and the second image on the first monitor screen.

6. The endotracheal intubation system of claim 1, wherein:
(i) the display means is electronically connected to the first camera means via a wired electronic connection, a wireless electronic connection, or both; and
(ii) the display means is electronically connected to the second camera means via a wired electronic connection, a wireless electronic connection, or both.

7. The endotracheal intubation system of claim 1, wherein the display means is mounted on the handle of the endotracheal intubation device so that the first view screen and the second view screen are viewable together by the operator during use.

8. The endotracheal intubation system of claim 1, wherein the display means is mounted adjacent the proximal end of the laryngoscope blade so that the first view screen and the second view screen are viewable together by the operator during use.

9. The endotracheal intubation system of claim 1, wherein the display means is mounted on a structure other than the endotracheal intubation device or the laryngoscope blade so that the first view screen and the second view screen are viewable together by the operator during use.

10. The endotracheal intubation system of claim 1, wherein:
(i) the endotracheal intubation device further comprises an operator handle mounted to the stylet at the proximal end of the stylet; and
(ii) the stylet is curveable at the distal end of the stylet adjacent the patient's airway passage by a translating means mounted on the operator handle and connected to the distal end of the stylet to curve the distal end of the stylet.

11. The endotracheal intubation system of claim 10, wherein the handle of the endotracheal intubation device comprises a trigger for squeezing by one hand of the operator to actuate the translating means and curve the distal end of the stylet while the operator's other hand manipulates the laryngoscope blade.

12. The endotracheal intubation system of claim 1, wherein the first camera means and the second camera means are independently selected from the group consisting of a CMOS device and a CCD device.

13. The endotracheal intubation system of claim 1, wherein the laryngoscope blade is curved to depress the patient's epiglottis with one side of the laryngoscope blade.

14. A method for placing an endotracheal tube in a patient by an operator, the method comprising:
(a) providing the endotracheal intubation system of claim 1, the endotracheal intubation system further comprising an endotracheal tube mounted to the stylet;
(b) inserting the laryngoscope blade into the patient's airway passage and manipulating the laryngoscope blade controlled by one hand of the operator to lift the patient's epiglottis while viewing the second image of the patient's airway passage during insertion and manipulation of the laryngoscope blade; and
(c) placing the endotracheal tube in the patient's airway passage with the endotracheal intubation device controlled by another hand of the operator while viewing the first image of the patient's airway passage during placement of the endotracheal tube.

15. The method of claim 14, wherein
(i) inserting and manipulating the laryngoscope blade into the patient's airway passage defines an open passageway between a surface of the laryngoscope blade and surrounding soft tissue in the patient's airway passage; and
(ii) placing the endotracheal tube in the patient's airway passage comprises inserting the endotracheal tube through the open passageway and into the to the patient's trachea.

16. The method of claim 14, wherein placing the endotracheal tube in the patient's airway passage comprises advancing at least a distal portion of the endotracheal tube beyond the distal end of the laryngoscope blade and within a field of view of the second camera means while viewing the second image of the endotracheal tube during advancement of the endotracheal tube.

17. The method of claim 16, wherein placing the endotracheal tube in the patient's airway passage comprises advancing the endotracheal tube into the patient's trachea while viewing the second image of the endotracheal tube during advancement of the endotracheal tube to control a degree of insertion of the endotracheal tube into the trachea.

18. The method of 17, wherein (i) the endotracheal tube comprises externally visible length-indicating means thereon, and (ii) controlling the degree of insertion of the endotracheal tube comprises viewing the second image of the length-indicating means during advancement of the endotracheal tube into the trachea.

19. The method of 17, wherein (i) the endotracheal tube comprises externally visible length-indicating means thereon, (ii) inserting the laryngoscope blade into the patient's airway passage comprises advancing the blade to a position where the patient's vocal cords are at least partially within the field of view of the second camera means, and (iii) controlling the degree of insertion of the endotracheal tube comprises viewing the second image of the length-indicating means at a position above the vocal cords during advancement of the endotracheal tube into the trachea to attain a preselected linear insertion distance of the distal end of the endotracheal tube into the trachea and relative to the vocal cords.

20. The method of claim 19, wherein the linear insertion distance is at least about 2 cm.

21. The method of claim 19, wherein the linear insertion distance ranges between about 3 cm and about 5 cm.

22. The method of claim 14, wherein the display means comprises one or more monitor screens adapted to independently and simultaneously display the first image and the second image.

23. The method of claim 16, wherein the one or more monitor screens are LCD screens.

24. The method of claim 14, wherein the display means comprises (i) a first monitor screen comprising the first view screen and being adapted to display the first image, and (ii) a second monitor screen comprising the second view screen and being adapted to display the second image.

25. The method of claim 14, wherein the display means comprises a first monitor screen, the first monitor screen comprising the first view screen and the second view screen as discrete regions of the first monitor screen and being adapted to independently and simultaneously display the first image and the second image on the first monitor screen.

26. The method of claim 14, wherein:
(i) the display means is electronically connected to the first camera means via a wired electronic connection, a wireless electronic connection, or both; and
(ii) the display means is electronically connected to the second camera means via a wired electronic connection, a wireless electronic connection, or both.

27. The method of claim 14, wherein the display means is mounted on the handle of the endotracheal intubation device so that the first view screen and the second view screen are viewable together by the operator during use.

28. The method of claim 14, wherein the display means is mounted adjacent the proximal end of the laryngoscope blade so that the first view screen and the second view screen are viewable together by the operator during use.

29. The method of claim 14, wherein the display means is mounted on a structure other than the endotracheal intubation device or the laryngoscope blade so that the first view screen and the second view screen are viewable together by the operator during use.

30. The method of claim 14, wherein the stylet is curveable at the distal end of the stylet adjacent the patient's airway passage by a translating means mounted on the operator handle and connected to the distal end of the stylet to curve the distal end of the stylet.

31. The method of claim 30, wherein the handle of the endotracheal intubation device comprises a trigger for squeezing by one hand of the operator to actuate the translating means and curve the distal end of the stylet while the operator's other hand manipulates the laryngoscope blade.

32. The method of claim 14, wherein the first camera means and the second camera means are independently selected from the group consisting of a CMOS device and a CCD device.

33. The method of claim 14, wherein the laryngoscope blade is curved to depress the patient's epiglottis with one side of the laryngoscope blade.
